SEQUENCE LISTING (28 sequences)

SEQ ID NO: 1 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 2 - DNA, 1455 bp, Mus sp.
cccgggaattcgccaccatggactggatctggcggatcctgttcctcgtgggagccgccacaggcgcccattctgcccagcccgccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacaccagccggctgcacagcggcgtgcccagcagattttctggcagcggcagcggcaccgactacagcctgaccatctccaacctggaacaggaagatatcgctacctacttctgtcagcaaggcaacaccctgccctacaccttcggcggaggcaccaagctggaactgaagagaggcggcggaggctctggtggaggcggatctgggggcggaggaagtggcgggggaggatctgaagtgcagctgcagcagagcggccctggcctggtggcccctagccagagcctgtccgtgacctgtaccgtgtccggcgtgtccctgcccgactacggcgtgtcctggatccggcagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgtccagcgccctgtccaacagcatcatgtacttcagccacttcgtgcccgtgtttctgcccgccaagcccaccaccacccctgcccctagacctcccaccccagccccaacaatcgccagccagcctctgtccctgcggcccgaagctagcagacctgctgccggcggagccgtgcacaccagaggcctggaccccaagctgtgctacctgctggacggcatcctgttcatctatggcgtgatcctgaccgccctgttcctgagagtgaagttcagcagaagcgccgacgcccctgcctaccagcagggccagaaccagctgtacaacgagctgaacctgggcagacgggaagagtacgacgtgctggacaagcggagaggcagggaccccgagatgggcggcaagcccagacggaagaacccccaggaaggcctgtataacgaactgcagaaagacaagatggccgaggcctacagcgagatcggcatgaagggcgagcggcggaggggcaagggccacgatggactgtaccagggcctgagcaccgccaccaaggacacctacgacgccctgcacatgcaggccctgccccccagatgacagccagggcatttctccctcgagcggccgc

SEQ ID NO: 3 - protein, 468 aa, Mus sp.
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSALSNSIMYFSHFVPVFLPAKPTTTPAPRPPTPAPTIASQPLSLRPEASRPAAGGAVHTRGLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 813 bp, Artificial Sequence
  source: /note=“Description of Artificial Sequence Synthetic polynucleotide”
atggactggatctggcggattctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatct

SEQ ID NO: 5 - protein, 271 aa, Artificial Sequence
  source: /note=“Description of Artificial Sequence Synthetic polypeptide”
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 6 - DNA, 807 bp, Artificial Sequence
  source: /note=“Description of Artificial Sequence Synthetic polynucleotide”
atggactggatctggcgcatcctcttcctcgtcggcgctgctaccggcgctcattcggcccagccggccatggcgcaagtaaaactccaagaatctggggcggagctggtgaaaccgggggcgtctgtgaagatgagctgtaaagcatcaggctacaccttcacctcctataatatgcactgggtgaaacaaacacccggacagggcctcgaatggattggtgccatctatcctggaaatggtgatacctcatataatcagaagtttaagggcaaggctacgcttactgcggataaaagctcttccactgcttacatgcaactgagcagtctcacttcagaggactcagccgattattattgtgcccgcagcaactactatggtagttcatactggtttttcgacgtttgggggcaaggtaccaccgtcacggtttcttctggtgggggcggaagcgggggtggaggatctgggggcggtggttcagacattgaactcacccagagccctactattctgagcgcgtctccaggtgaaaaagttacgatgacgtgcagagcatcaagtagtgtgaattatatggattggtatcaaaagaagccaggctcatccccaaaaccgtggatctatgcaactagcaacctcgcgtcaggggtgccagcaaggttttccggaagtggttctggcacatcttatagtctcaccatttcccgagtggaggctgaggatgcggccacttattactgccagcaatggtcattcaatcccccaacatttggtggcggaacaaaactcgaaattaaacgg

SEQ ID NO: 7 - protein, 269 aa, Artificial Sequence
  source: /note=“Description of Artificial Sequence Synthetic polypeptide”
MDWIWRILFLVGAATGAHSAQPAMAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKR

SEQ ID NO: 8 - DNA, 795 bp, Artificial Sequence
  source: /note=“Description of Artificial Sequence Synthetic polynucleotide”
atggactggatctggcgcatcctcttcctcgtcggcgctgctaccggcgctcattcggcccagccggccgacattcaaatgactcagtccccttccagcttgtcagcctcagtaggggaccgggtcacgatcacctgtcgagcgtctgagtcagtggataactacgggatttctttcatgaactggttccagcagaagcccggcaaagctcctaagctccttatatatgcagcctcaaatcaggggagcggtgttcctagtcgcttcagtggaagcggtagcggtacggactttacgttgacgataagtagccttcagccagatgactttgccacttattattgtcagcagtctaaggaagttccttggacgtttggccaaggaacgaaggtcgaaatcaaagggggagggggctcaggagggggcggcagtggtggtggaggctctcaagtccaactcgtacagtctggcgcggaggttaaaaagccgggaagctccgtgaaagtatcctgtaaggcaagcggatacacctttaccgattataacatgcactgggttaggcaggcgcccggccaaggtctggaatggatcggttatatttatccatacaacggtggtaccggctataatcagaagtttaagagtaaggctactattacagcggatgagtcaaccaatactgcatacatggagctctcctcactcaggagcgaagataccgcagtgtattactgtgcccgagggagaccagccatggactactggggtcagggtacccttgtgacagtatctagc

SEQ ID NO: 9 - protein, 265 aa, Artificial Sequence
  source: /note=“Description of Artificial Sequence Synthetic polypeptide”
MDWIWRILFLVGAATGAHSAQPADIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 10 - DNA, 840 bp, Artificial Sequence
  source: /note=“Description of Artificial Sequence Synthetic polynucleotide”
atggactggatctggcgcatcctcttcctcgtcggcgctgctaccggcgctcattcggcccagccggccgatatcgagctcacccaatctccaaaattcatgtccacatcagtaggagacagggtcagcgtcacctgcaaggccagtcagaatgtggatactaatgtagcgtggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggcggcaccaagctggaaatcaaacgggctgccgcagaaggtggaggcggttcaggtggcggaggttccggcggaggtggctctggcggtggcggatcggccatggcccaggtgaagctgcagcagtcaggagggggcttggtgcaacctggaggctccatgaaactctcctgtgttgtctctggattcactttcagtaattactggatgaactgggtccgccagtctccagagaaggggcttgagtggattgcagaaattagattgaaatccaataattttggaagatattatgcggagtctgtgaaagggaggttcaccatctcaagagatgattccaaaagtagtgcctacctgcaaatgatcaacctaagagctgaagatactggcatttattactgtaccagttatggtaactacgttgggcactattttgaccactggggccaagggaccacggtcaccgtatcgagt

SEQ ID NO: 11 - protein, 280 aa, Artificial Sequence
  source: /note=“Description of Artificial Sequence Synthetic polypeptide”
MDWIWRILFLVGAATGAHSAQPADIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAAAEGGGGSGGGGSGGGGSGGGGSAMAQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 12 - DNA, 807 bp, Artificial Sequence
  source: /note=“Description of Artificial Sequence Synthetic polynucleotide”
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcg

SEQ ID NO: 13 - protein, 269 aa, Artificial Sequence
  source: /note=“Description of Artificial Sequence Synthetic polypeptide”
MDWIWRILFLVGAATGAHSAQPADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 14 - DNA, 822 bp, Artificial Sequence
  source: /note=“Description of Artificial Sequence Synthetic polynucleotide”
atggactggatctggcggattctgtttctcgtgggagctgccacaggcgctcattctgctcagcctgccgatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaaggcggcggaggaagcggaggcggaggatctgggggcggaggctctggcggagggggatctcaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatcc

SEQ ID NO: 15 - protein, 274 aa, Artificial Sequence
  source: /note=“Description of Artificial Sequence Synthetic polypeptide”
MDWIWRILFLVGAATGAHSAQPADVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSS

SEQ ID NO: 16 - DNA, 822 bp, Artificial Sequence
  source: /note=“Description of Artificial Sequence Synthetic polynucleotide”
atggactggatctggcggattctgtttctcgtgggagctgccacaggcgctcattctgctcagcctgccgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagggcggcggaggaagcggaggcggaggatctgggggcggaggctctggcggagggggatctgaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagc

SEQ ID NO: 17 - protein, 274 aa, Artificial Sequence
  source: /note=“Description of Artificial Sequence Synthetic polypeptide”
MDWIWRILFLVGAATGAHSAQPADIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKGGGGSGGGGSGGGGSGGGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSS

SEQ ID NO: 18 - DNA, 825 bp, Artificial Sequence
  source: /note=“Description of Artificial Sequence Synthetic polynucleotide”
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 19 - protein, 275 aa, Artificial Sequence
  source: /note=“Description of Artificial Sequence Synthetic polypeptide”
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 20 - DNA, 798 bp, Artificial Sequence
  source: /note=“Description of Artificial Sequence Synthetic polynucleotide”
atggactggatctggcggattctgtttctcgtgggagctgccacaggcgctcattctgctcagcctgccagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaaggcggcggaggaagcggaggcggaggatctgggggcggaggctctggcggagggggatctcaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatct

SEQ ID NO: 21 - DNA, 798 bp, Artificial Sequence
  source: /note=“Description of Artificial Sequence Synthetic polynucleotide”
atggactggatctggcggattctgtttctcgtgggagctgccacaggcgctcattctgctcagcctgcccaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatctggcggcggaggaagcggaggcggaggatctgggggcggaggctctggcggagggggatctagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaa

SEQ ID NO: 22 - protein, 266 aa, Artificial Sequence
  source: /note=“Description of Artificial Sequence Synthetic polypeptide”
MDWIWRILFLVGAATGAHSAQPASIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIKGGGGSGGGGSGGGGSGGGGSQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSS

SEQ ID NO: 23 - protein, 266 aa, Artificial Sequence
  source: /note=“Description of Artificial Sequence Synthetic polypeptide”
MDWIWRILFLVGAATGAHSAQPAQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSSGGGGSGGGGSGGGGSGGGGSSIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIK

SEQ ID NO: 24 - protein, 254 aa, Unknown
  source: /note=“Description of Unknown High Affinity Variant Immunoglobulin Gamma Fc Region Receptor III-A amino acid sequence”
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 25 - DNA, 765 bp, Unknown
  source: /note=“Description of Unknown High Affinity Variant Immunoglobulin Gamma Fc Region Receptor III-A nucleic acid sequence”
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 26 - protein, 61 aa, Homo sapiens
LSNSIMYFSHFVPVFLPAKPTTTPAPRPPTPAPTIASQPLSLRPEACRPAAGGAVHTRGLD

SEQ ID NO: 27 - DNA, 183 bp, Homo sapiens
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggat

SEQ ID NO: 28 - protein, 135 aa, Homo sapiens
PKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2021/0187024 A1
(43) Pub. Date: Jun. 24, 2021

(54) NK-92 Cells to Stimulate Anti-Cancer Vaccine

(71) Applicant: NantKwest, Inc., San Diego, CA (US)

(72) Inventors: Hans G. Klingemann, Boston, MA (US); Laurent H. Boissel, Brookline, MA (US); Nathan Schomer, Culver City, CA (US)

(21) Appl. No.: 16/760,893

(22) PCT Filed: Oct. 31, 2018
(86) PCT No.: PCT/US2018/058535

§ 371 Date: Apr. 30, 2020

Related U.S. Application Data
(60) Provisional application No. 62/579,975, filed on Nov. 1, 2017.
(60) Provisional application No. 62/628,683, filed on Feb. 9, 2018.

(51) Int. Cl. A61K 35/17; A61P 35/00; A61K 38/20; A61K 45/06; C07K 16/28; A61K 39/395; C07K 14/705; C07K 14/725; A61K 38/17

(52) U.S. Cl. CPC A61K 35/17; A61P 35/00; A61K 38/2013; A61K 38/208; A61K 45/06; C07K 16/2803; A61K 2039/505; C07K 14/70517; C07K 14/7051; A61K 38/1774; C07K 2319/30; C07K 2319/33; A61K 39/3955

(57) ABSTRACT

Provided herein are methods for inducing and maintaining an immune response to a tumor in a subject while treating a primary tumor. The methods include administering to the subject an effective amount of CAR-expressing-NK-92 cells to treat the primary tumor thereby inducing an anti-tumor immune response that is maintained in the subject, the maintained immune response preventing tumor regrowth and/or inhibiting generation of secondary tumors. Also provided are methods of producing an anti-tumor vaccine in a subject with a tumor. The methods include administering to the subject an effective amount of CAR-expressing-NK-92 cells to the subject thereby inducing an anti-tumor vaccine to the tumor in the subject.

### CROSS REFERENCE TO RELATED APPLICATIONS

This application claims benefit of priority to U.S. Provisional Application No. 62/579,975 filed Nov. 1, 2017, and U.S. Provisional Application No. 62/628,683 filed Feb. 9, 2018, each of which is hereby incorporated by reference in its entirety.

### SEQUENCE LISTING

The instant application contains a Sequence Listing which has been filed electronically in ASCII format and is hereby incorporated by reference in its entirety. Said ASCII copy, created on Oct. 30, 2018, is named 104066-1111776_SL.txt and is 50,729 bytes in size.

### BACKGROUND OF THE INVENTION

Cancer is a leading cause of illness and death worldwide. For example, over 1.5 million new cancer cases and more than half a million cancer deaths are projected to occur in the United States in 2015. While several cancer therapies exist, all have serious drawbacks.

Chemotherapy involves the disruption of cell replication or cell metabolism, and it remains one of the main treatment options for cancer. Chemotherapy can be effective, but there are severe side effects, e.g., vomiting, low white blood cells (WBC), loss of hair, loss of weight and other toxic effects. Because of the extremely toxic side effects, many cancer individuals cannot successfully finish a complete chemotherapy regime. Cancer drug monotherapy also selects for mutant cancer cells that are resistant to the drug.

One traditional alternative/adjunct to chemotherapy is radiation therapy. Radiation therapy uses high-energy radiation to damage tumor cells' DNA, causing them to stop proliferating and/or die. However, radiation is non-specific and kills healthy cells along with the cancerous ones. Targeted radiation (e.g., external beam radiation, brachytherapy) only targets specific, known tumors in the patient, whereas systemic radiation has a greater potential of harming a large number of normal cells and tissues. Radiation also has negative side effects, including a risk of a secondary cancer caused by the radiation.

Advances in immunotherapy poses some benefits and involves the use of certain cells of the immune system that have cytotoxic activity against particular target cells. Natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells have been shown to be somewhat effective in both ex vivo therapy and in vivo treatment. NK cells used for this purpose are isolated from the peripheral blood lymphocyte (“PBL”) fraction of blood from the subject, expanded in cell culture in order to obtain sufficient numbers of cells, and then re-infused into the subject. However, such therapy is complicated by the fact that not all NK cells are cytolytic and the therapy is specific to the treated patient.

Due to the severity and prevalence of cancer, there is still a great need for effective treatments of such diseases or disorders that overcome the shortcomings of current treatments.

### SUMMARY OF THE INVENTION

Provided herein are methods for inducing and maintaining an immune response to a tumor in a subject while treating a primary tumor. The methods include administering to the subject an effective amount of CAR-expressing-NK-92 cells to treat the primary tumor thereby inducing an anti-tumor immune response that is maintained in the subject, the maintained immune response preventing tumor regrowth and/or inhibiting generation of secondary tumors. Also provided are methods of producing an anti-tumor vaccine in a subject with a tumor. The methods include administering to the subject an effective amount of CAR-expressing-NK-92 cells to the subject thereby inducing an anti-tumor vaccine to the tumor in the subject.

In one aspect, described herein is a method for inducing and maintaining an immune response to a tumor in a subject while treating a primary tumor. In some embodiments, the method comprises administering to the subject an effective amount of CAR-expressing-NK-92 cells to treat the primary tumor thereby inducing an anti-tumor immune response that is maintained in the subject, the maintained immune response preventing tumor regrowth and/or inhibiting generation of secondary tumors.

In some embodiments, the method results in interleukin 6 expression being increased in the subject.

In some embodiments, the CAR-expressing-NK-92 cells induce lysis of tumor cells in the primary tumor.

In some embodiments, a cytokine is co-administered to the subject. In some embodiments, the cytokine is interleukin 2. In some embodiments, the cytokine is interleukin 12.

In some embodiments, a chemotherapeutic agent is administered to the subject. In one embodiment, the chemotherapeutic agent is administered to the subject prior to administration of the CAR-expressing-NK-92 cells. In one embodiment, the chemotherapeutic agent is administered to the subject after administration of the CAR-expressing-NK-92 cells. In one embodiment, the chemotherapeutic agent is administered to the subject substantially simultaneously with administration of the CAR-expressing-NK-92 cells.

In some embodiments, the CAR-expressing-NK-92 cells are administered systemically. In some embodiments, the CAR-expressing-NK-92 cells are administered proximate to or directly into the primary tumor.

In some embodiments, the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor. In some embodiments, the tumor is a B-cell lymphoma.

In some embodiments, the method further comprises administering to the subject a cancer drug or radiation.

In some embodiments, the subject is selected from the group consisting of bovines, swine, rabbits, alpacas, horses, canines, felines, ferrets, rats, mice, fowl and buffalo. In one embodiment, the subject is a human.

In some embodiments, the CAR-expressing-NK-92 cells express a CD19-CAR on the cell surface.

In some embodiments, the NK-92 cell is modified to express a chimeric antigen receptor (CAR) on the cell surface. In some embodiments, the CAR comprises an antigen binding domain (e.g., ScFv) that specifically binds an antigen expressed by tumor cells. In one embodiment, the antigen binding domain specifically binds the CD19 antigen. In some embodiments, the tumor cells comprise lymphoma cells. In some embodiments, the NK-92 cells express a CAR that specifically binds CD19 and the tumor cells comprise lymphoma cells. In some embodiments, the NK-92 cells express murine CD19CAR (mCD19CAR) on the cell surface. In one embodiment, the mCD19CAR comprises an amino acid sequence having at least 90% identity to SEQ ID NO: 3. In some embodiments, the NK-92 cells express a codon optimized CAR on the cell surface, where the CAR is codon optimized for expression in humans. In one embodiment, the NK-92 cells express a codon optimized CD19CAR on the cell surface. In one embodiment, the codon optimized CD19CAR comprises an amino acid sequence at least 90% identical to SEQ ID NO: 5.

In one embodiment, the NK-92 cells express a codon optimized CD20CAR on the cell surface. In one embodiment, the codon optimized CD20CAR comprises an amino acid sequence at least 90% identical to SEQ ID NO: 7. In one embodiment, the NK-92 cells express a codon optimized CD33CAR on the cell surface. In one embodiment, the codon optimized CD33CAR comprises an amino acid sequence at least 90% identical to SEQ ID NO: 9. In one embodiment, the NK-92 cells express a codon optimized CSPG4-CAR on the cell surface. In one embodiment, the codon optimized CSPG4-CAR comprises an amino acid sequence at least 90% identical to SEQ ID NO: 11. In one embodiment, the NK-92 cells express a codon optimized EGFR-CAR on the cell surface. In one embodiment, the codon optimized EGFR-CAR comprises an amino acid sequence at least 90% identical to SEQ ID NO: 13. In one embodiment, the NK-92 cells express a codon optimized IGF1R-CAR on the cell surface. In one embodiment, the codon optimized IGF1R-CAR comprises an amino acid sequence at least 90% identical to SEQ ID NO: 15. In one embodiment, the NK-92 cells express a codon optimized CD30-CAR on the cell surface. In one embodiment, the codon optimized CD30-CAR comprises an amino acid sequence at least 90% identical to SEQ ID NO: 17. In one embodiment, the NK-92 cells express a codon optimized HER2/neu-CAR on the cell surface. In one embodiment, the codon optimized HER2/neu-CAR comprises an amino acid sequence at least 90% identical to SEQ ID NO: 19. In one embodiment, the NK-92 cells express a codon optimized GD2-CAR on the cell surface. In one embodiment, the codon optimized GD2-CAR comprises an amino acid sequence at least 90% identical to SEQ ID NO: 22 or SEQ ID NO:23.

In another aspect, described herein is a method of producing an anti-tumor vaccine in a subject with a tumor, the method comprising administering to the subject an effective amount of CAR-expressing-NK-92 cells thereby inducing an anti-tumor vaccine to the tumor in the subject.

In some embodiments, the method results in increased expression of interleukin 6 in the subject.

In some embodiments, the CAR-expressing-NK-92 cells treats the tumor in the subject.

In some embodiments, a cytokine is co-administered to the subject. In some embodiments, the cytokine is interleukin 2. In some embodiments, the cytokine is interleukin 12.

In some embodiments, a chemotherapeutic agent is administered to the subject. In one embodiment, the chemotherapeutic agent is administered to the subject prior to administration of the CAR-expressing-NK-92 cells. In one embodiment, the chemotherapeutic agent is administered to the subject after administration of the CAR-expressing-NK-92 cells. In one embodiment, the chemotherapeutic agent is administered to the subject substantially simultaneously with administration of the CAR-expressing-NK-92 cells.

In some embodiments, the CAR-expressing-NK-92 cells are administered systemically. In some embodiments, the CAR-expressing-NK-92 cells are administered proximate to or directly into the primary tumor.

In some embodiments, the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor. In some embodiments, the tumor is a B-cell lymphoma.

In some embodiments, the method further comprises administering to the subject a cancer drug or radiation.

In some embodiments, the subject is selected from the group consisting of bovines, swine, rabbits, alpacas, horses, canines, felines, ferrets, rats, mice, fowl and buffalo. In one embodiment, the subject is a human.

In some embodiments, the CAR-expressing-NK-92 cells are mCD19CAR-expressing NK-92 cells.

In another aspect, provided is a CAR-expressing-NK-92 cell for use in treating a primary or secondary tumor in a subject. In some embodiments, provided is a CAR-expressing-NK-92 cell for use in inducing and maintaining an immune response to a tumor in a subject while treating a primary tumor. In some embodiments, the use comprises administering to the subject an effective amount of CAR-expressing-NK-92 cells to treat the primary tumor thereby inducing an anti-tumor immune response that is maintained in the subject, the maintained immune response preventing tumor regrowth and/or inhibiting generation of secondary tumors.

### BRIEF DESCRIPTION OF THE FIGURES

FIG. 1 is a graph showing that wild type NK-92 cells produce IL-8, IL-10, and interferon gamma (IFNγ), but not assayable amounts of IL-6 as determined by qualitative ELISA assay.

FIG. 2A shows surface expression of mCD19CAR in cells in flow cytometry experiments. FIG. 2B shows killing of murine A20 lymphoma cells in vitro by mCD19CAR-expressing NK-92 cells.

FIG. 3 is a graph showing reduced tumor surface area (mm²) after NK-92-CD19CAR administration (circles) and continued regression until the tumor is no longer visible. Tumor surface area initially reduces after injection of wild type NK-92 cells (stars), but subsequently increases and tumor regrows.

FIGS. 4A-4D show intra-tumor treatment promotes clearance of A20 tumor tumors and increases survival. FIG. 4A is a schematic showing the experimental methodology. FIG. 4B is a stacked bar graph depicting the percentage of tumor seeding observed in each condition. No statistically significant differences were found when a two-tailed Fisher's exact test was used to compare efficiency of tumor seeding between females and males. FIG. 4C shows the change in tumor volume over time in separate graphs for each of the treatments: vehicle, parental NK-92 cells, or mCD19CAR-NK-19 cells. Each male and female for each treated group are plotted separately. FIG. 4D is a graph of a Kaplan-Meyer curve detailing cumulative survival of study animals. Animals euthanized due to tumors exceeding 1500 mm³ or tumors that were ulcerated were counted towards survival analysis. Data were analyzed by log-rank (Mantel-Cox) test. *=p<0.05.

FIG. 5 is a bar graph showing average tumor volumes for males, females, or both on Day 16 post-treatment.

FIG. 6 is a graph showing survival of mice after re-challenge with A20 tumor cells. All tumor-free mice surviving by Day 30 were re-challenged by subcutaneous injection of A20 cells in the contralateral flank. All mice remained tumor-free and survived until day 60 post-treatment, except one.

FIG. 7 shows a Kaplan-Meier survival curve of mice injected with A20 tumor cells following intratumor treatment with mCD19-CAR NK-92 cells vs. vehicle control, as described in the Examples.

FIG. 8 shows tumor size of complete responders vs. naïve controls re-challenged with A20 tumor cells, as described in the Examples.

FIG. 9 shows a Kaplan-Meier survival curve of mice injected with L1210-Luc tumor cells following intratumor treatment with mCD19-CAR NK-92 cells vs. vehicle control, as described in the Examples.

FIG. 10 shows tumor size of complete responders vs. naïve controls re-challenged with L1210-Luc tumor cells, as described in the Examples.

### DETAILED DESCRIPTION OF THE INVENTION

After reading this description, it will become apparent to one skilled in the art how to implement the methods and compositions in various alternative embodiments and alternative applications. It will be understood that the methods and compositions presented here are presented by way of an example only, and not limitation. It is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this disclosure belongs.

In this specification and in the claims that follow, reference will be made to a number of terms that shall be defined to have the following meanings:

The terminology used herein is for the purpose of describing particular embodiments only and is not intended to be limiting of the subject matter claimed. As used herein, the singular forms “a,” “an” and “the” are intended to include the plural forms as well, unless the context clearly indicates otherwise.

It is understood that all numerical values described herein (e.g., pH, temperature, time, concentration, amounts, and molecular weight, including ranges) include normal variation in measurements encountered by one of ordinary skill in the art. Thus, numerical values described herein include variation of +/−0.1 to 10%, for example, +/−0.1%, 0.5%, 1%, 2%, 3%, 4%, 5%, 6%, 7%, 8%, 9%, or 10%. It is to be understood, although not always explicitly stated, that all numerical designations may be preceded by the term “about.” It is also to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed subject matter. “Consisting of” shall mean excluding more than trace amount of other ingredients and substantial method steps. Embodiments defined by each of these transition terms are within the scope of this disclosure.

As used herein, “concurrent” or “concurrently” refers to the administration of at least two agents (e.g. NK-92-Fc-CAR cells and a cancer drug) at the same time or at approximately the same time

The term “cancer drugs” refers to chemical and biological agents used to treat cancer. Such cancer drugs include, but are not limited to, chemotherapeutic agents, hormonal therapy agents, and the like as well as combinations thereof.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In a particularly preferred embodiment, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. The term “administering” or “administration” of a monoclonal antibody or a natural killer cell to a subject includes any route of introducing or delivering the antibody or cells to perform the intended function. Administration can be carried out by any route suitable for the delivery of the cells or monoclonal antibody. Thus, delivery routes can include intravenous, intramuscular, intraperitoneal, or subcutaneous deliver. In some embodiments a monoclonal antibody and/or NK-92 cells are administered directly to the tumor, e.g., by injection into the tumor. Administration includes self-administration and the administration by another.

The term “effective dose” or “effective amount” refers to a dose of an agent or composition (e.g., NK-92 cells) containing the agent that produces the desired effect(s) (e.g., treating or preventing a disease). The exact dose and formulation will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington (2012); and Pickar, Dosage Calculations (9th edition) (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several seconds, minutes, hours, or days before administering the other active ingredient or ingredients.

The term “simultaneous” therapeutic use refers to the administration of at least two active ingredients by the same or different route and at the same time or at substantially the same time.

The term “primary tumor” generally refers to the original tumor. Cells from the primary tumor may break off and form secondary. As a practical matter, the primary tumor is a known tumor that is desired to be treated by the cancer drugs and/or NK-92 cell therapy. In a preferred embodiment, the primary tumor is located at the site of origin for the cancer. For example, a primary tumor for a breast cancer is located in the breast.

The term “secondary tumor,” “metastasis,” or “metastatic tumor” as used herein refers to a tumor that is related to (e.g., arose/metastasized from) the primary tumor but located at a site distinct from the primary tumor. For example, a secondary tumor for breast cancer may be located in the bone. Secondary tumor formation is a problem for cancer treatment.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92™, NK-92™-CD16, NK-92™-CD16-γ, NK-92™-CD16-ζ, NK-92™-CD16(F176V), NK-92™MI, and NK-92™CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+ NK-92™ cells” or “haNK® cells”). In some embodiments, the CD16+ NK-92™ cells comprise a high affinity CD16 receptor on the cell surface. The high affinity CD16 molecule contains a phenylalanine to valine substitution at codon/position 158 (F158V) of the mature CD16 peptide, which binds with higher affinity to human IgG1 than does CD16 with phenylalanine (F) at codon 158. The term “taNK” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”). The term “t-haNK” refers to natural killer cells derived from the highly potent unique cell line described in Gong et al. (1994), rights to which are owned by NantkWest, modified to express CD 16 on the cell surface and to express a chimeric antigen receptor (hereafter, “CAR-modified CD16+ NK-92™ cells” or “t-haNK™ cells”). In some embodiments, the t-haNK™ cells express a high affinity CD16 receptor on the cell surface.

The original NK-92 cell line expressed the CD56^{bright}, CD2, CD7, CD11a, CD28, CD45, and CD54 surface markers. The original NK-92 cell line does not display the CD1, CD3, CD4, CD5, CD8, CD10, CD14, CD16, CD19, CD20, CD23, and CD34 markers. Growth of NK-92 cells in culture is typically dependent upon the presence of interleukin 2 (rIL-2), with a dose as low as 1 IU/mL being sufficient to maintain proliferation. NK-92 cells have high cytotoxicity even at a low effector:target (E:T) ratio, e.g., 1:1. (Gong, et al., supra).

A “modified NK-92 cell” refers to an NK-92 cell that expresses an exogenous gene or protein, such as an Fc receptor, a CAR, a cytokine (such as IL-2 or IL-12), and/or a suicide gene. In some embodiments, the modified NK-92 cell comprises a vector that encodes for a transgene, such as an Fc receptor, a CAR, a cytokine (such as IL-2 or IL-12), and/or a suicide gene. In one embodiment, the modified NK-92 cell expresses at least one transgenic protein.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be inactivated by another mechanism.

As used herein, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

As used herein, the terms “cytotoxic” and “cytolytic,” when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

The term “kill” with respect to a cell/cell population is directed to include any type of manipulation that will lead to the death of that cell/cell population.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92 cells do not express FcγRIII-A. A representative polynucleotide sequence encoding a native form of CD16 is shown in SEQ ID NO:1. The high affinity Fc Receptor III-A amino acid sequence (full length) is shown in SEQ ID NO:24.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific antigens. Examples of CARs and/or scFv domains include those that recognize the following antigens: CD19 (SEQ ID NO:3, SEQ ID NO:5), CD 20 (SEQ ID NO:7); CD33 (SEQ ID NO:9), CSPG4 (SEQ ID NO:11), EGFR (SEQ ID NO:13), IGF1R (SEQ ID NO:15), CD30 (SEQ ID NO:17), HER2/neu (SEQ ID NO:19), and GD2 (SEQ ID NO:22 (VL/VH format) or SEQ ID NO:23 (VH/VL format)).

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

As used herein, “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some embodiments, a homologous amino acid sequence has no more than 15, nor more than 10, nor more than 5 or no more than 3 conservative amino acid substitutions. In some embodiments, a nucleotide or amino acid sequence has at least 60%, at least 65%, at least 70%, at least 80%, or at least 85% or greater percent identity to a sequence described herein. In some embodiments, a nucleotide or amino acid sequence has at least 90%, 91%, 92%, 93%, 94%, 95%, 96%, 97%, 98% or 99% identity to a sequence described herein. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). Algorithms suitable for determining percent sequence identity include the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at ncbi.nlm.nih.gov). The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

The term “expression” refers to the production of a gene product. The term “transient” when referred to expression means a polynucleotide is not incorporated into the genome of the cell.

The term “cytokine” or “cytokines” refers to the general class of biological molecules which effect cells of the immune system. Exemplary cytokines include, but are not limited to, interferons and interleukins (IL), in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “antibody” refers to an immunoglobulin or fragment thereof. The antibody may be of any type (e.g., IgG, IgA, IgM, IgE or IgD). Preferably, the antibody is IgG. An antibody may be non-human (e.g., from mouse, goat, or any other animal), fully human, humanized, or chimeric.

As used herein, the term “antibody fragment” refers to any portion of the antibody that recognizes an epitope. Antibody fragments may be glycosylated. By way of non-limiting example, the antibody fragment may be a Fab fragment, a Fab′ fragment, a F(ab′)2 fragment, a Fv fragment, an rIgG fragment, a functional antibody fragment, single chain recombinant forms of the foregoing, and the like. F(ab′)2, Fab, Fab′ and Fv are antigen-binding fragments that can be generated from the variable region of IgG and IgM. They vary in size, valency, and Fc content. The fragments may be generated by any method, including expression of the constituents (e.g., heavy and light chain portions) by a cell or cell line, or multiple cells or cell lines. Preferably, the antibody fragment recognizes the epitope and contains a sufficient portion of an Fc region such that it is capable of binding an Fc receptor.

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

As used herein, the term “anti-tumor vaccine” refers to the induction and maintenance of an immune response to a tumor preventing tumor regrowth and/or generation of secondary tumors.

Titles or subtitles may be used in the specification for the convenience of a reader, which are not intended to influence the scope of the claimed subject matter. Additionally, some terms used in this specification are more specifically defined below.

Due to concerns that NK-92 cells might proliferate in the body and cause unwanted side effects, these cells can be irradiated prior to administration to the patient. Irradiated NK-92 cells survive only about 24 to 48 hours after administration to the patient. As the NK-92 cells are likely to target the primary tumor and have limited half-lives, metastatic cells and cancer stem cells may elude this treatment. However, as described herein, administration of NK-92 cells induces an immune response in the subject that is able to produce an anti-tumor vaccine, and that such response persists in the subject after the NK-92 cells have died. Further, as described herein, administration of NK-92 cells induces an immune response in a subject that is capable of rejecting a tumor upon tumor re-challenge. Thus, administration of NK-92 cells at or near the site of a tumor, specifically, CAR expressing NK-92 cells, acts as a vaccine against the tumor. Thus, CAR-expressing-NK-92 are capable of preventing tumor regrowth.

The CAR-expressing NK-92 cells, when administered, are sufficient to treat a primary tumor while also eliciting an immune response that prevents potential secondary tumors and/or tumor regrowth. For example, an effective amount of NK-92 cells results in lysis of at least a portion of tumor cells in the primary tumor, and also causes the patient's immune system to recognize antigens from the tumor such that tumor cells are recognized and attacked (e.g., by T cells) even after the NK-92 cells are no longer active in the patient. The therapeutically effective amount of the CAR-expressing NK-92 cells will vary depending on the tumor being treated and its severity as well as the age, weight, etc., of the patient to be treated. The skilled artisan will be able to determine appropriate dosages depending on these and other factors. The compositions can also be administered in combination with one or more additional therapeutic compounds.

Without being bound by theory, it is believed that administration of NK-92 cells to treat a primary tumor in a first location can lead to a prolonged anti-tumor immune response prevents secondary tumors (metastases) at other locations in the patient's body, even after the NK-92 cells have ceased to function. Thus, provided is a method of treating cancer in a subject comprising administering to the subject an effective amount of NK-92 cells to induce an immune response in the subject, the immune response is capable of inhibiting generation of secondary tumors.

NK-92 cells do not express interleukin 6 (IL-6). IL-6 is a marker of increased immune response in a patient. In one embodiment, IL-6 expression is increased in the patient after administration of NK-92 cells. In one embodiment, IL-6 expression persists after the administered NK-92 cells cease to function.

The tumor may be, for example, a colorectal tumor, a breast tumor, a lung tumor, a prostate tumor, a pancreatic tumor, a bladder tumor, a cervical tumor, cholangiocarcinoma, gastric sarcoma, glioma, leukemia, lymphoma, melanoma, multiple myeloma, osteosarcoma, an ovarian tumor, a stomach tumor, a brain tumor.

Optionally, one or more additional cancer treatments or therapies are administered to the subject to treat the primary tumor. Optionally, a cancer drug is administered to the subject. Optionally, radiation is administered to the subject. Where additional cancer treatments are administered, they may be administered prior to, concurrently with, and/or after administration of the CAR-expressing NK-92 cells.

### NK-92 Cells

The NK-92 cell line is a unique cell line that was discovered to proliferate in the presence of interleukin 2 (IL-2). Gong et al., Leukemia 8:652-658 (1994). These cells have high cytolytic activity against a variety of cancers. The NK-92 cell line is a homogeneous cancerous NK cell population having broad anti-tumor cytotoxicity with predictable yield after expansion. Phase I clinical trials have confirmed its safety profile. NK-92 was discovered in the blood of a subject suffering from a non-Hodgkins lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells nor do they elicit an unacceptable immune rejection response in humans. Characterization of the NK-92 cell line is disclosed in WO 1998/49268 and U.S. Patent Application Publication No. 2002-0068044.

As described herein, NK-92 cells can be further engineered to express a chimeric antigen receptor (CAR) on the cell surface. Optionally, the CAR is specific for a tumor-specific antigen. Tumor-specific antigens are described, by way of non-limiting example, in US 2013/0189268; WO 1999024566 A1; U.S. Pat. No. 7,098,008; and WO 2000020460 A1, each of which is incorporated herein by reference in its entirety. Tumor-specific antigens include, without limitation, CD19, CD20, NKG2D ligands, CS1, GD2, CD138, EpCAM, HER-2, EBNA3C, GPA7, CD244, CA-125, MUC-1, ETA, MAGE, CEA, CD52, CD30, MUC5AC, c-Met, EGFR, FAB, WT-1, PSMA, NY-ESO1, CSPG-4, IGF1-R, Flt-3, CD276, BCMA, CD33, or 41BB. Optionally, the CAR is a CD19 CAR. Representative polynucleotide and polypeptide sequences for the CD19 CAR are provided in SEQ ID NO:2 and SEQ ID NO:4 (CD19 CAR polynucleotide), and SEQ ID NO:3 and SEQ ID NO:5 (CD19 CAR polypeptide).

In some embodiments, the CAR comprises an ScFv antigen-binding domain. In some embodiments, the CAR comprises an antigen binding domain (e.g., ScFv) that specifically binds an antigen expressed by tumor cells. In some embodiments, the antigen binding domain or ScFv specifically binds the following antigens: CD19, CD20, NKG2D ligands, CS1, GD2, CD138, EpCAM, HER-2, EBNA3C, GPA7, CD244, CA-125, MUC-1, ETA, MAGE, CEA, CD52, CD30, MUC5AC, c-Met, EGFR, FAB, WT-1, PSMA, NY-ESO1, CSPG-4, IGF1-R, Flt-3, CD276, BCMA, CD33, or 41BB. In one embodiment, the antigen binding domain or ScFv specifically binds the CD19 antigen. In some embodiments, the CAR comprises an antigen binding domain or ScFv having the following sequences (or a sequence at least 90%, 91%, 92%, 93%, 94%, 95%, 96%, 97%, 98% or 99% identical to the following sequences): CD19 (SEQ ID NO:3, SEQ ID NO:5), CD 20 (SEQ ID NO:7); CD33 (SEQ ID NO:9), CSPG4 (SEQ ID NO:11), EGFR (SEQ ID NO:13), IGF1R (SEQ ID NO:15), CD30 (SEQ ID NO:17), HER2/neu (SEQ ID NO:19), and GD2 (SEQ ID NO:22 (VL/VH format) or SEQ ID NO:23 (VH/VL format)).

In some embodiments, the CAR comprises a hinge region from CD8. In some embodiments, the hinge region comprises the amino acid sequence of SEQ ID NO: 26, or an amino acid sequence at least 90%, 91%, 92%, 93%, 94%, 95%, 96%, 97%, 98% or 99% identical to SEQ ID NO: 26. In some embodiments, the CAR comprises a transmembrane domain from CD3zeta. In some embodiments, the transmembrane domain comprises SEQ ID NO: 28, or an amino acid sequence at least 90%, 91%, 92%, 93%, 94%, 95%, 96%, 97%, 98% or 99% identical to SEQ ID NO: 28.

In some embodiments, the NK-92 cell or cell line is genetically modified with a nucleic acid construct that encodes a CAR described herein. In some embodiments, the nucleic acid construct further comprises a promoter that promotes transcription of the nucleic acid sequences. In some embodiments, the promoter is an inducible promoter. In some embodiments, the nucleic acid construct comprises a nucleic acid sequence that encodes an antigen binding protein (ABP). In some embodiments, the ABP is an scFv or a codon optimized scFv. In some embodiments, the ABP specifically binds an antigen expressed by a tumor cell. In some embodiments, the ABP comprises a region of a CAR described herein (in other words, the CAR comprises the ABP). In some embodiments, the construct comprises a nuclei acid that encodes a cytokine, such a IL-2. In one embodiment, the cytokine is targeted to the endoplasmic reticulum.

In one embodiment, the CAR is transiently expressed by the NK-92 cell. In one embodiment, the CAR is stably expressed by the NK-92 cell.

Optionally, NK-92 cells are modified to express an Fc receptor protein on the cell surface. Exemplary, non-limiting Fc receptors include CD64, CD32, CD16 (e.g., CD16a and CD16b), FcεRI, CD23, CD89, Fcα/μR, and FcRn. In some embodiments, the Fc receptor is CD16. In some embodiments, the Fc receptor is a high-affinity Fc receptor comprising a valine at position 158 of the mature protein, or a valine at the position corresponding to position 158 of the mature protein (CD16 F158V). In some embodiments, when the modified NK-92 cells express an Fc receptor, an antibody specific for the target tumor cell is co-administered with the NK-92 cells. Co-administration encompasses administration of the antibody immediately prior to, concurrently with, or immediately after administration of the NK-92 cells.

Illustrative Fc receptors are shown in the following Table:

TABLE X
Illustrative Fc receptors

| | Principal | Affinity | | |
|---|---|---|---|---|
| | antibody | for | | Effect following binding |
| Receptor name | ligand | ligand | Cell distribution | to antibody |
| FcγRI (CD64) | IgG1 and | High | Macrophages | Phagocytosis |
| | IgG3 | (Kd~10⁻⁹M) | Neutrophils | Cell activation |
| | | | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > 10⁻⁷M) | Neutrophils | Degranulation (eosinophils) |
| | | | Eosinophils | |
| | | | Platelets | |
| | | | Langerhans cells | |
| FcγRIIB1 (CD32) | IgG | Low | B Cells | No phagocytosis |
| | | (Kd > 10⁻⁷M | Mast cells | Inhibition of cell activity |
| FcγRIIB2 (CD32) | IgG | Low | Macrophages | Phagocytosis |
| | | (Kd > 10⁻⁷M) | Neutrophils | Inhibition of cell activity |
| | | | Eosinophils | |
| FcγRIIIA (CD16a) | IgG | Low | NK cells | Induction of antibody- |
| | | (Kd > 10⁻⁶M) | Macrophages | dependent cell-mediated |
| | | | (certain tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > 10⁻⁶M) | Macrophages | killing |
| | | | Neutrophils | |
| | | | Mast cells | |
| | | | Follicular dendritic | |
| | | | cells | |
| FcϵRI | IgE | High | Mast cells | Degranulation |
| | | (Kd~10⁻¹⁰M) | Eosinophils | Phagocytosis |
| | | | Basophils | |
| | | | Langerhans cells | |
| | | | Monocytes | |
| FcϵRII (CD23) | IgE | Low | B cells | Possible adhesion molecule |
| | | (Kd > 10⁻⁷M) | Eosinophils | IgE transport across human |
| | | | Langerhans cells | intestinal epithelium |
| | | | | Positive-feedback |
| | | | | mechanism to enhance |
| | | | | allergic sensitization |
| | | | | (B cells) |
| FcαRI (CD89) | IgA | Low | Monocytes | Phagocytosis |
| | | (Kd > 10⁻⁶M | Macrophages | Induction of microbe |
| | | | Neutrophils | killing |
| | | | Eosinophils | |
| Fcα/μR | IgA and | High for | B cells | Endocytosis |
| | IgM | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to infant in milk |
| | | | Hepatocytes | Protects IgG from |
| | | | | degradation |

Optionally, NK-92 cells are modified to express at least one cytokine. In particular, the at least one cytokine is IL-2, IL-12, IL-15, IL-18, IL-21, or a variant thereof. In preferred embodiments, the cytokine is IL-12 or a variant thereof. In especially preferred embodiments, the cytokine is IL-2 or a variant thereof. In certain embodiments, the cytokine is a variant that is targeted to the endoplasmic reticulum.

NK-92 cells can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive. In other embodiments, NK-92 cells can be administered to such an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth)NK-92 cells per kilogram of the individual, or any ranges between any two of the numbers, end points inclusive. In other embodiments, the total dose may calculated by m² of body surface area, including 1×10¹¹, 1×10¹⁰, 1×10⁹, 1×10⁸, 1×10⁷, per m². The average person is 1.6-1.8 m².

### Methods of Treatment

Provided herein are methods for inducing and maintaining an immune response to a tumor in a subject. Optionally, the methods include treating a primary tumor while inducing and maintaining an immune response to the tumor in the subject. The methods include administering to the subject an effective amount of CAR-expressing-NK-92 cells to treat the primary tumor thereby inducing an anti-tumor immune response that is maintained in the subject. The maintained immune response prevents tumor regrowth and/or inhibits generation of secondary tumors. Optionally, interleukin 6 expression is increased in the patient. Optionally, the CAR-expressing-NK-92 cells induce lysis of tumor cells in the primary tumor. Optionally, a cytokine is co-administered to the subject. Optionally, the cytokine is interleukin 2. Optionally, the cytokine is interleukin 12. Optionally, a chemotherapeutic agent is administered to the subject prior to administration of the CAR-expressing-NK-92 cells. Optionally, the CAR-expressing-NK-92 cells are administered systemically. Optionally, the CAR-expressing-NK-92 cells are administered proximate to or directly into the primary tumor. Optionally, the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor. Optionally, the method further includes administering to the subject a cancer drug or radiation to the patient. Optionally, the subject is selected from the group consisting of bovines, swine, rabbits, alpacas, horses, canines, felines, ferrets, rats, mice, fowl and buffalo. Optionally, the subject is human. Optionally, the CAR-expressing-NK-92 cells are mCD19CAR-expressing NK-92 cells. Optionally, the tumor is a B-cell lymphoma.

Also provided are methods of producing an anti-tumor vaccine in a subject with a tumor comprising administering to the subject an effective amount of CAR-expressing-NK-92 cells to the subject thereby inducing an anti-tumor vaccine to the tumor in the subject. Optionally, interleukin-6 expression is increased in the subject Optionally, the CAR-expressing-NK-92 cells treats the tumor in the subject. Optionally, a cytokine is co-administered to the subject. Optionally, the cytokine is interleukin 2. Optionally, the cytokine is interleukin 12. Optionally, a chemotherapeutic agent is administered to the subject prior to administration of the CAR-expressing-NK-92 cells. Optionally, the CAR-expressing-NK-92 cells are administered systemically. Optionally, the CAR-expressing-NK-92 cells are administered proximate to or directly into the tumor. Optionally, the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor. Optionally, the method further includes administering to the subject a cancer drug or radiation. Optionally, the CAR-expressing-NK-92 cells are mCD19CAR-expressing NK-92 cells. Optionally, the tumor is a B-cell lymphoma.

Optionally, the CAR-expressing NK-92 cells are administered systemically to the subject, e.g. by intravenous injection. Optionally, the CAR-expressing NK-92 cells are administered locally to the site of a tumor, e.g., intraperitoneal administration or injection of NK-92 cells proximate to or directly into the tumor. Benefits of local administration of CAR-expressing NK-92 cells include, but are not limited to, the ability to use fewer cells to obtain an effect and an increased concentration of CAR-expressing NK-92 cells at the tumor site.

Optionally, a cytokine or multiple cytokines are administered to the subject concurrently with the CAR-expressing NK-92 cells. Optionally, the cytokine is a cytokine that further stimulates an immune response. Optionally, the cytokine is a cytokine that elicits a T cell and/or NK cell response. Optionally, the cytokine is IL-2 and/or IL-12. Without being bound by theory, it is believed that administration of cytokines will further elicit a T cell response against the tumor and/or potentiate CAR-expressing NK-92 cell activity. Optionally, the cytokine is administered systemically to the patient. Optionally, the cytokine is administered locally to the site of the primary tumor.

Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

NK-92 cells can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive.

Optionally, said individual can be administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) NK-92 cells per injection, or any ranges between any two of the numbers, end points inclusive.

Optionally, NK-92 cells can be administered to such individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive.

Optionally, the total dose may calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient. In other embodiments, the amount of NK-92 cells injected per dose may calculated by m² of body surface area, including 1×10¹¹, 1×10¹⁰, 1×10⁹, 1×10⁸, 1×10⁷, per m². The average person is 1.6-1.8 m².

The NK-92 cells, and optionally other anti-cancer drugs (e.g., chemotherapeutic agents) can be administered once to a patient with cancer can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

Optionally, NK-92 cells are administered in a composition comprising NK-92 cells and a medium, such as human serum or an equivalent thereof. Optionally, the medium comprises human serum albumin. Optionally, the medium comprises human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. In Optionally, a serum substitute that is acceptable for use in human therapeutics is used instead of human serum. Such serum substitutes are known in the art. Although concentrations of human serum over 15% can be used, it is contemplated that concentrations greater than about 5% will be cost-prohibitive. Optionally, NK-92 cells including modified NK-92 cells (e.g., CAR-expressing NK-92 cells) are administered in a composition comprising NK-92 cells and an isotonic liquid solution that supports cell viability. Optionally, NK-92 cells are administered in a composition that has been reconstituted from a cryopreserved sample.

Pharmaceutically acceptable compositions can include a variety of carriers and excipients. A variety of aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

These compositions for use in in vivo or in vitro may be sterilized by conventional, well-known sterilization techniques. The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

Optionally, the NK-92 cells are administered to the patient in conjunction with one or more other treatments for the cancer being treated. Without being bound by theory, it is believed that co-treatment of a patient with NK-92 cells and another therapy for the cancer will allow the NK-92 cells and the alternative therapy to give the endogenous immune system a chance to clear the cancer that heretofore had overwhelmed such endogenous action. I Optionally, two or more other treatments for the cancer being treated includes, for example, an antibody, radiation, chemotherapeutic, stem cell transplantation, or hormone therapy.

Optionally, an antibody is administered to the patient in conjunction with the NK-92 cells. In one embodiment, the NK-92 cells and an antibody are administered to the patient together, e.g., in the same formulation; separately, e.g., in separate formulations, concurrently; or can be administered separately, e.g., on different dosing schedules or at different times of the day. When administered separately, the antibody can be administered in any suitable route, such as intravenous or oral administration.

In the provided methods of treatment, additional therapeutic agents can be used that are suitable to the disease being treated. Thus, in some embodiments, the provided methods of treatment further comprise administering a second therapeutic agent to the subject. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are for illustrative purposes only and should not be interpreted as limitations of the claimed subject matter. There are a variety of alternative techniques and procedures available to those of skill in the art which would similarly permit one to successfully perform the claimed subject matter.

### Example 1

### NK-92 Cytokine Production In Vitro

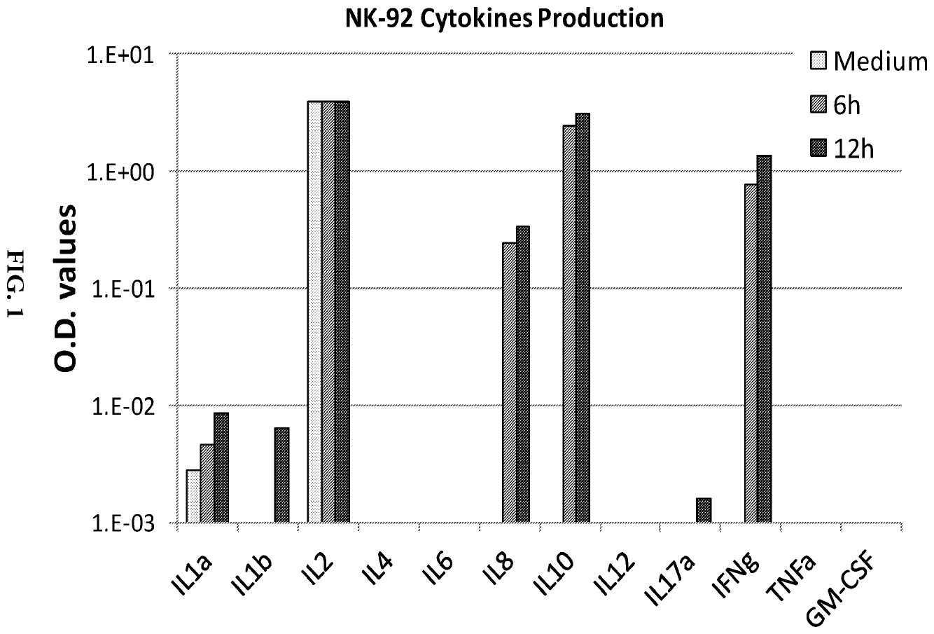

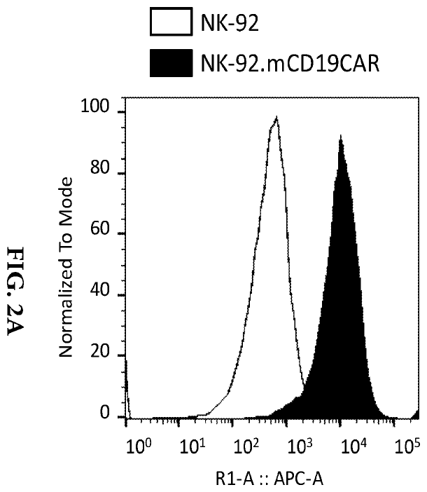

Wild type NK-92 production of a variety of cytokines was determined by qualitative ELISA assay. Results are shown in FIG. 1. NK-92 cells produce IL-8, IL-10, and interferon gamma (IFNγ). NK-92 cells do not produce assayable amounts of IL-6.

### Example 2

### Expression and In Vitro Activity of mCD19CAR in NK-92

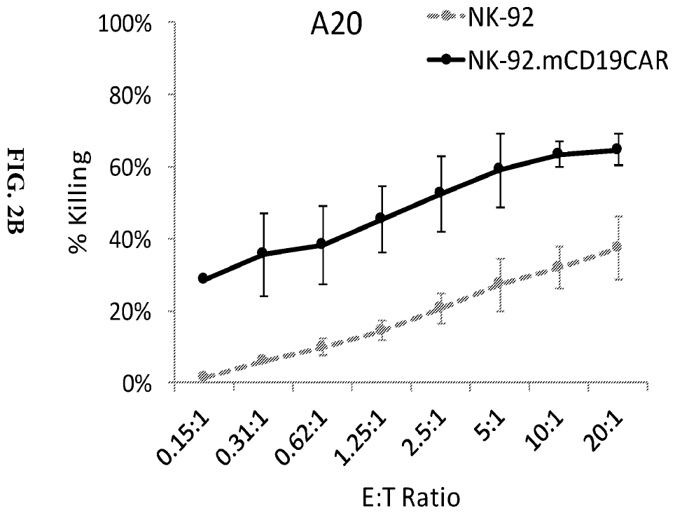

NK-92 cells were transduced with a retrovirus coding for a second generation anti-murine CD19-CAR. Significant amounts of CD19-CAR were detectable by flow cytometry in whole NK-92 cells (FIG. 2A). mCD19CAR-NK-92 cells were added to A20 murine lymphoma cells at effector:target (E:T) ratios ranging from 0.15:1 to 20:1. At each E:T ratio tested, mCD19CAR-NK-92 cells killed a greater percentage of A20 lymphoma cells as compared to wild type NK-92 cells (FIG. 2B). For both NK-92 cell types, increased E:T ratios resulted in increased killing of A20 lymphoma cells. These results show that CD92-CAR is highly expressed in NK-92 cells, and that mCD19CAR-NK-92 cells is more effective at killing A20 lymphoma cells compared to wild-type NK-92 cells in vitro.

### Example 3

### In Vivo Development of Immune Response After Localized Administration of NK-92 Cells in Mice

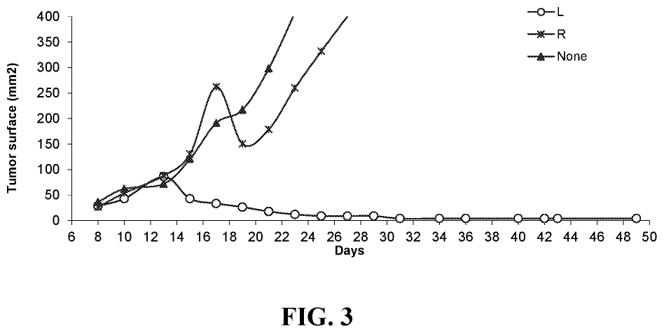

In preliminary experiments, Balb-c mice were injected (subcutaneous) with A20 murine lymphoma cells (A20 are murine CD19⁺). After tumor was established (at approximately days 6 to 8), mice were injected intra-tumor either with a control or NK-92 cells (FIG. 3: PBS control, triangle; wild type, star; CD19CAR, circle) at days 13 and 15 post-lymphoma cell injection. The mouse administered CD19CAR was re-challenged twice with A20 murine lymphoma cells on the opposite flank from the original injection at approximately day 36 post-inoculation, and a second time on the original flank at day 54 post-inoculation. At the time of sacrifice (approximately 8 weeks after the second challenge) the necropsy showed no signs of tumors.

Results are shown in FIG. 3. Tumor surface area (mm²) was reduced after the first NK-92-CD19CAR administration and continued regression until the tumor was no longer visible. Tumor surface area was reduced after the second injection of wild type NK-92 cells, but not the first, and the tumor resumed growth within several days of the second NK-92 cell injection. Surprisingly, lymphoma re-challenge in the animal administered NK-92-CD19CAR did not result in tumor growth, regardless of the site of lymphoma cell injection, even though the NK-92-CD19CAR cells would be expected to have ceased activity in the mouse at the time of re-challenge.

### Example 4

### Antitumor Vaccination Using CD19-CAR-Expressing NK-92 Cells in Treatment of Subcutaneous A20 B-Cell Lymphoma in Balb/c Mice

The following example demonstrates NK-92 cells can be successfully redirected to specifically kill target cells from murine origin through expression of an anti-murine CAR. Intra-tumor injection of NK-92.mCD19CAR induces clearance of s.c. A20 lymphoma cell tumors and significantly improves survival. Successful tumor clearance correlates with resistance to later challenges with A20 cells, indicating the development of a long-term immune response in the treated mice. Resistance to A20 cells re-challenges appears to be independent on T-cells.

The long-term goal of cancer treatment is to achieve elimination of tumor cells anywhere in the body through induction of a specific immune memory response. In addition to spontaneous cytotoxicity, ADCC and cytokine release, NK cells also contribute to an adaptive immune response through crosstalk with dendritic cells and T-cells. To further characterize the NK cell-induced adaptive response, NK-92 cells (aNK) were transduced with a lentivirus construct coding for a third generation anti-murine CD19 CAR and injected intra-tumorally (5×10e6 NK cells, two injections three days apart) into a murine syngeneic subcutaneous lymphoma (10e6 A20 cells into BALB/c mice). Tumor size was monitored over time and mice showing clearance of the tumors were re-challenged with another subcutaneous injection of A20 cells contralaterally.

Targeted activated NK-92 cells (mCD19-CAR taNK) effectively killed murine cancer cells in vitro (>60% killing at E:T ratio of 5:1). In vivo, intra-tumor injection of mCD19CAR taNK induced significant tumor regression compared to saline (342 mm3 and 936 mm3 respectively at day 16, p<0.05) and significantly improved survival, with 75% of the mice showing complete tumor regression (p<0.05). In contrast, injection of parental NK cells did not significantly affect tumor size in mice (815 mm3 at day 16). Importantly, re-challenge of the tumor-free mice with A20 cells failed to induce tumor regrowth after 14 days in 5 out of 6 mice, suggesting induction of a memory (“vaccine”) effect after the injection of mCD19 taNK.

In conclusion, the human NK-92 cell expressing an anti-murine CD19-CAR (mCD19 taNK) can effectively kill CD19-positive murine cancer cells. Moreover, intra-tumor injections of targeted mCD19CAR taNK into a fully immunocompetent mouse model can induce tumor clearance and protection from tumor re-challenge.

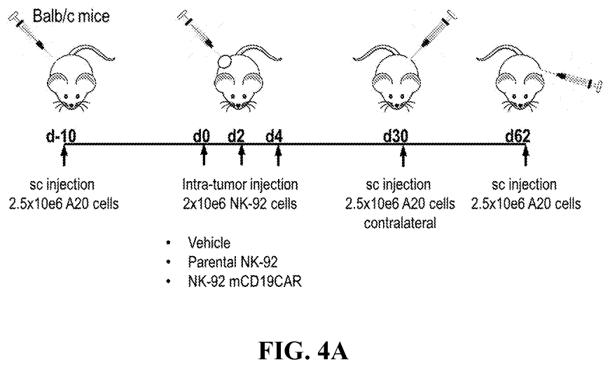

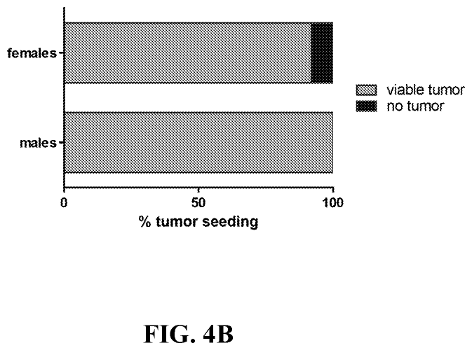

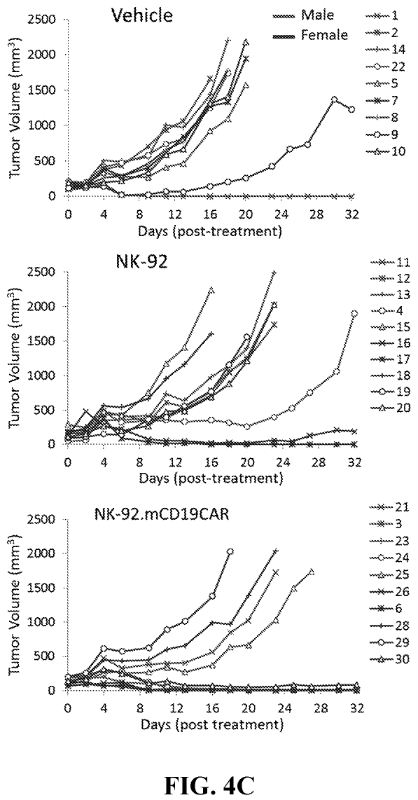

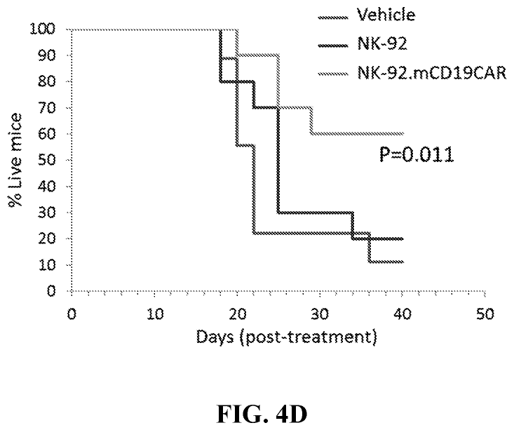

To establish these results, forty (40) Balb/c mice (20 males and 20 females) aged 5-6 weeks were enrolled in a study to determine the effects of intra-tumor treatment on tumor clearance and animal survival. All animals were housed under standard environmental conditions in groups of five (5) animals each of the same sex, with even numbered groups consisting of females and odd numbered groups consisting of males, and maintained on appropriate rodent chow and sterile water ad libitum. FIG. 4A is a schematic showing the experimental design. Mice were anesthetized with isoflurane and injected with 2.5×10⁶ A20 murine lymphoma cells in 100 μL volume of serum free media, subcutaneously (s.c.) into the left flank. Beginning two days after tumor cell inoculation, tumors were measured daily by digital caliper. Ten days after inoculation, single inoculation animals of the same sex were randomized around a mean tumor volume (˜140 mm³ at time of randomization) into Groups 1-6, with each group consisting of animals bearing a similar mean tumor volume and range. The day of randomization was considered Day 0 of the study, and intratumoral (i.t.) administration of test treatments in a volume of 50 μl serum-free RPMI-1640 media commenced for all animals on this day. Animals in Groups 1-2 were administered vehicle only (serum-free RPMI-1640). Animals in Groups 3-4 were administered NK-92 parental cells (NK-92.C). Animals in Groups 5-6 were administered mCD19-CAR-NK-92 cells. Following the Day 0, test treatment administration, identical i.t. cell treatments were performed on Day 2 and Day 4. Tumors were measured three times each week (3×/week) by digital caliper to monitor tumor growth, and animals were weighed and monitored daily for general health and survival, and assigned a Body Condition Score (see Body Condition Scoring) once a week (1×/week). Any animal bearing a tumor of volume ≥1500 mm³ or a tumor that has ulcerated; or any animal that has lost ≥30% of its body weight at Day 0; or displays a Body Condition Score of ≤2; or is moribund were euthanized by CO₂ overdose. Changes in tumor volumes for male and female mice treated with vehicle, NK-92, or mCD19CAR-NK-19 are plotted in FIG. 4C. Mouse survival is shown in FIG. 4D.

Animals in Groups 1-6 showing a complete response (i.e. elimination of s.c. tumor mass) were re-challenged with a second s.c. injection of 2.5×10⁶ A20 cells into the right flank (contralateral) on Day 30. These animals were monitored for tumor formation, and if tumors developed, these were measured by digital caliper 3×/week.

Animal gender did not significantly impact rates of tumor seeding/rejection of syngeneic Balb/c A20 lymphoma cells.

Treatment with mCD19-CAR NK-92 cells provided a statistically significant survival advantage to male mice bearing a single subcutaneous A20 tumor (p=0.0415).

A trend of enhanced survival was observed for mCD-19-CAR NK-92 treated females bearing single A20 tumors, however this difference fell short of statistical significance (p=0.085).

No statistically significant differences in cumulative weight change were observed when comparing parental or targeted NK-92 treated groups (Groups 3-6) to vehicle controls for male or female mice.

For male animals, single A20 tumors treated with mCD19-CAR-NK-92 cells (Group 5) displayed inhibited growth kinetics compared to vehicle treated tumors (Group 1). Cumulative differences in these tumor growth kinetics approached statistical significance (p=0.055).

For female animals, single A20 tumors treated with parental NK-92 cells (Group 4) or mCD19-CAR-NK-92 cells (Group 6) displayed inhibited growth kinetics compared to control. However, no statistically significant differences were detected when comparing cumulative tumor volume of any group to that of vehicle treated controls.

With the exception of a single female mouse previously treated with mCD19-CAR NK-92 cells, no tumor engraftment was observed in animals that had been previously successful in rejecting a tumor upon re-challenge, regardless of treatment or previous kinetics of tumor regression/rejection.

### Material and Methods

Location of Study Performance. The study was performed at Biomodels' facility in Watertown, Mass. IACUC approval (13-0627-2) for this study was obtained from Biomodels' IACUC. The Office of Laboratory Animal Welfare (OLAW) assurance number is A4591-01.

Animal identification. Male or female Balb/c mice (BALB/cAnNTac; Taconic Biosciences) aged 5-6 weeks, with mean body weight (±SD) of 21.65 g±2.47 on Day 0 were used. Animals were uniquely identified using an ear punch. Animals were acclimatized at least 3 days prior to study commencement. During this period, the animals were observed daily in order to reject animals that were in poor condition.

Housing. The study was performed in animal rooms provided with filtered air at a temperature of 70±5° F. and 50±20% relative humidity. Animals were housed in closed ventilation, HEPA-filtered disposable caging in small groups of 2-3 animals per cage. Animal rooms were set to maintain a minimum of 12 to 15 air changes per hour. The room was on an automatic timer for a light/dark cycle of 12 hours on and 12 hours off with no twilight. Sterile wood chip bedding or equivalent bedding was used. Bedding was changed a minimum of once per week. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity was recorded during the study, and the records retained.

Diet. Animals were maintained with LabDiet 5053 Rodent Diet and sterile water provided ad libitum.

Animal Randomization and Allocations. Animals were randomly and prospectively divided by sex into six (6) treatment groups of five (5) animals each, ten (10) days prior to tumor cell inoculation (on Day 0). Even numbered Groups consisted of females and odd numbered Groups consisted of males. On Day 0, single inoculation animals (Groups 1-6) were randomized around the mean tumor volume (˜135 mm³ at time of randomization), by sex, with each group consisting of animals bearing a similar mean tumor volume and range. On Day 0, mean tumor volumes (mm³±SEM) for enrolled animals from each group (N=5) were as follows: Group 1: 149.22±50.20; Group 2: 128.02±64.94; Group 3: 158.68±95.54; Group 4: 129.73±30.60; Group 5: 149.58±65.50; Group 6: 128.46±48.71.

Cell Culture/Inoculation. A20 cells were provided by the Sponsor. Cells were counted by hemocytometer/trypan-blue exclusion and 2.5×10⁶ A20 murine lymphoma cells in 100 μL volume of serum free media injected subcutaneously (s.c.) into the left flank.

Intratumoral Injections. Animals were injected intratumorally (i.t.) with 2×10⁶ vehicle, parental NK-92, or mCD19-CAR-NK92 cells in 50 μl volume on Days 0, 2 and 4 as indicated in Table 1. Actual cell numbers injected were as follows: Day 0: NK-92=800,000; mCD19-CAR-NK92=700,000; Day 2: NK-92=1.3×10⁶; mCD19-CAR-NK92=1.3×10⁶; Day 4: NK-92=1.34×10⁶; mCD19-CAR-NK92=1.3×10⁶. Cells were delivered by 25 G needle, inserted so that the tip was at the approximate center of the tumor mass. The cells were slowly released into the tumor, and the needle held in place for a minimum of 30 seconds following dose to allow for the volume to be absorbed into the tumor. The tumor was squeezed between forceps at the entry site as the needle was slowly withdrawn to prevent leakage of the dose from the entry. The forceps were held in place following needle exit for an additional 30 seconds.

Experimental Design. Forty (40) Balb/c mice (20 males and 20 females; Taconic Biosciences) aged 5-6 weeks were enrolled in the study. All animals were housed under standard environmental conditions in groups of five (5) animals each of the same sex, with even numbered Groups consisting of females and odd numbered Groups consisting of males, and maintained on appropriate rodent chow and sterile water ad libitum. Mice were anesthetized with isoflurane and injected with 2.5×10⁶ A20 murine lymphoma cells in 100 μL volume of serum free media, subcutaneously (s.c.) into the left flank. Beginning on two days after tumor cell inoculation, tumors were measured daily by digital caliper. Ten days after inoculation, single inoculation animals of the same sex were randomized around a mean tumor volume (˜140 mm³ at time of randomization) into Groups 1-6, with each groups consisting of animals bearing a similar mean tumor volume and range. The day of randomization was considered Day 0 of the study, and intratumoral (i.t.) administration of test treatments in a volume of 50 μl serum-free RPMI-1640 media commenced for all animals on this day. Animals in Groups 1-2 were administered vehicle only (serum-free RPMI-1640). Animals in Groups 3-4 were administered NK-92 parental cells (NK-92.C). Animals in Groups 5-6 were administered mCD19-CAR-NK-92 cells. Following the Day 0 test treatment administration, identical i.t. cell treatments were performed on Day 2 and Day 4. Tumors were measured three times each week (3×/week) by digital caliper to monitor tumor growth, and animals were weighed and monitored daily for general health and survival, and assigned a Body Condition Score (see Body Condition Scoring) once a week (1×/week). Any animal bearing a tumor of volume ≥1500 mm³ or a tumor that has ulcerated; or any animal that has lost ≥30% of its body weight at Day 0; or displays a Body Condition Score of >2; or is moribund will be euthanized by CO₂ overdose.

Animals in Groups 1-6 showing a complete response (i.e. elimination of s.c. tumor mass) were re-challenged with a second s.c. injection of 2.5×10⁶ A20 cells into the right flank on Day 30. These animals were monitored for tumor formation, and if tumors developed, these were measured by digital caliper 3×/week. The experimental details of the in-life portion of the study described above are outlined in Table 1.

TABLE 1
Tumor Response Study Design

| | | Cell Line | Cell Line | Treatment | | |
|---|---|---|---|---|---|---|
| | Number | # injected | # injected | # injected | | |
| | of | (left | (right | (left flank | Tumor Volume | |
| Group | Animals | flank) | flank) | only) | (digital caliper) | Endpoint |
| 1 | 5/male | A20 | — | Vehicle | Pre- | Study termination |
| | | 2.5 × 10⁶ | | (50 u1) | Randomization | (scheduled-Day 50) |
| | | (100 u1) | | Intratumor | Daily | weight loss ≥30%; |
| | | | | d0, d2, d4 | Randomized | BC Score <2; |
| | | | | | 3x/week | tumor volume ≥1500 mm³ |
| | | | | | (Mon, Wed, Fri) | |
| 2 | 5/female | A20 | — | Vehicle | Pre- | Study termination |
| | | 2.5 × 10⁶ | | (50 u1) | Randomization | (scheduled-Day 50) |
| | | (100 u1) | | Intratumor | Daily | weight loss ≥30%; |
| | | | | d0, d2, d4 | Randomized | BC Score <2; |
| | | | | | 3x/week | tumor volume ≥1500 mm³ |
| | | | | | (Mon, Wed, Fri) | |
| 3 | 5/male | A20 | — | NK-92 | Pre- | Study termination |
| | | 2.5 × 10⁶ | | parental | Randomization | (scheduled-Day 50) |
| | | (100 u1) | | (NK-92.C) | Daily | weight loss ≥30%; |
| | | | | 2 × 10⁶ | Randomized | BC Score <2; |
| | | | | (50 u1) | 3x/week | tumor volume ≥1500 mm³ |
| | | | | Intratumor | (Mon, Wed, Fri) | |
| | | | | d0, d2, d4 | | |
| 4 | 5/female | A20 | — | NK-92 | Pre- | Study termination |
| | | 2.5 × 10⁶ | | parental | Randomization | (scheduled-Day 50) |
| | | (100 u1) | | (NK-92.C) | Daily | weight loss ≥30%; |
| | | | | 2 × 10⁶ | Randomized | BC Score <2; |
| | | | | (50 u1) | 3x/week | tumor volume ≥1500 mm³ |
| | | | | Intratumor | (Mon, Wed, Fri) | |
| | | | | d0, d2, d4 | | |
| 5 | 5/male | A20 | — | mCD19- | Pre- | Study termination |
| | | 2.5 × 10⁶ | | CAR NK-92 | Randomization | (scheduled-Day 50) |
| | | (100 u1) | | 2 × 10⁶ | Daily | weight loss ≥30%; |
| | | | | (50 u1) | Randomized | BC Score <2; |
| | | | | Intratumor | 3x/week | tumor volume ≥1500 mm³ |
| | | | | d0, d2, d4 | (Mon, Wed, Fri) | |
| 6 | 5/female | A20 | — | mCD19- | Pre- | Study termination |
| | | 2.5 × 10⁶ | | CAR NK-92 | Randomization | (scheduled-Day 50) |
| | | (100 u1) | | 2 × 10⁶ | Daily | weight loss ≥30%; |
| | | | | (50 u1) | Randomized | BC Score <2; |
| | | | | Intratumor | 3x/week | tumor volume ≥1500 mm³ |
| | | | | d0, d2, d4 | (Mon, Wed, Fri) | |

Tumor Seeding. Tumor seeding efficiency was assessed on Day 0, prior to randomization. Any animal not developing a tumor exceeding 50 mm³ by study completion was excluded from the study. Such tumors were considered inviable.

Animal Survival. Animals were monitored for survival daily. Any animal requiring euthanasia according to animal health and welfare thresholds, including loss of greater than 30% of their initial body weight, tumors exceeding 1500 mm³ or ulcerated, inability to obtain food/water or moribund was included for representation of survival and statistical analysis.

Animal Weights. All animals were weighed daily. Group weight change was expressed as a mean percent weight change. Animals that lost greater than 30% of their total starting body weight were euthanized.

Tumor Measurement. Tumors were measured three times each week (3×/week) using a digital caliper. Tumor volume was calculated using the standard equation where volume (V)=L×W²/2. (L=tumor length W=tumor width). Animals were sacrificed when tumors ulcerated or reached a max volume of 1500 mm³. To preserve graph integrity for reporting, tumor volumes were carried over past sacrifice if necessary for graphing purposes until 50% of the animals in a Group were sacrificed, and the tumor volume analysis was discontinued at this point for each Group.

Body Condition Scoring. A Body Condition Score was assigned to all animals 1×/week, using the following criteria:

Body Condition Scoring Table

| | |
|---|---|
| BC5 | The animal is obese, smooth and bulky. One is unable to |
| | identify its bone structure under the flesh and fat. Often |
| | mice in this condition cannot groom well and hair coat |
| | may appear oily and stained. |
| BC4 | The animal is over-conditioned and vertebrae are only |
| | palpable with firm pressure. |
| BC3 | The animal is well-conditioned. Vertebrae and pelvis are |
| | palpable with light pressure. |
| BC2 | The animal is under-conditioned. Segmentation of the |
| | vertebral column is evident and pelvis is palpable. |
| BC1 | The animal is emaciated, skeletal structure very prominent |
| | with little flesh cover. Vertebrae are distinctly segmented. |

Animals Found Dead or Moribund. Animals were monitored on a daily basis and those exhibiting weight loss greater than 30%, were unable to ambulate, attain food and water, and/or appeared moribund were euthanized. Furthermore, if the tumors appeared ulcerated, or exceed 1500 mm³ the animal was euthanized. Animals requiring sacrifice were euthanized by CO₂ overdose and underwent necropsy to determine the presence of absence of tumors. Any adverse effects or unanticipated deaths were reported to the veterinarian and to the client immediately.

Statistical Analyses. Statistical differences between groups were determined using Fisher's exact test for tumor seeding efficiencies, Gehan-Breslow-Wilcoxon Chi-square test for cumulative survival, and by one-way ANOVA followed by Dunnett's post hoc test and/or Student's T-test for mean weight change, and tumor volume. Area under the curve analysis was performed to evaluate the animal's change in weight or tumor volume over the full duration of the study. Statistical significance was considered achieved at P<0.05.

### Results and Discussion

Tumor Seeding/Rejection. FIG. 4A shows the experimental design. Tumor seeding was assessed on Day 0, and again at study completion. Any tumor that did not exceed of a volume of 50 mm³ was considered inviable. FIG. 4B shows tumor take rate following subcutaneous injection of 2.5×10⁶ A20 Balb/c murine lymphoma cells into Balb/c mice was 100% (25/25) for male mice and 92% (23/25) for female mice. This difference was not statistically significant when tested by two-tailed Fisher's exact test. These observations suggest that animal gender does not significantly impact tumor seeding/rejection in this model.

Animal Survival. Animals were monitored for survival daily through Day 40. All animal deaths in the study were euthanizations due to tumors exceeding animal welfare thresholds of 1500 mm³ volumes or ulceration. Groups were considered by gender, with changes in tumor volume shown in FIG. 4C. Survival of mice is shown in FIG. 4D.

Of the tumor-bearing male mice receiving vehicle control treatments (Group 1), 20% (1/5) survived through Day 40. Of the males receiving NK-92 parental treatments (NK.92.C, Group 3), 0% (0/5) survived through Day 40, with the last animal in the group surviving until Day 39. Of the males bearing a single tumor and receiving mCD-19-CAR NK-92 treatments (Group 5), 60% (3/5) survived through Day 40. The Gehan-Breslow-Wilcoxon Chi-square test was used to test for statistically significant differences in overall survival between groups. The mCD19-CAR NK-92 treated animals bearing a single tumor (Group 5) displayed significantly enhanced survival compared to vehicle treated animals (p=0.0415). Survival was not statistically different in comparing parental NK-92 (Group 3) tumor bearing males to vehicle control animals (Group 1).

Of the tumor-bearing female mice receiving vehicle control treatments (Group 2), 0% (0/5) survived through Day 40, with the last animal in the group surviving until Day 38. Of the females receiving NK-92 parental treatments (NK.92.C, Group 4), 20% (1/5) survived through Day 40. Of the females bearing a single tumor and receiving mCD-19-CAR NK-92 treatments (Group 6), 60% (3/5) survived through Day 40. Survival was not statistically different in comparing any treatment group of tumor bearing females to vehicle control animals (Group 1).

These results indicate that treatment with mCD19-CAR NK-92 cells provides a statistically significant survival advantage to male mice bearing a single subcutaneous A20 tumor (p=0.0415). A trend of enhanced survival was also observed for mCD-19-CAR NK-92 females bearing a single A20 tumor, however this difference fell short of statistical significance (p=0.085).

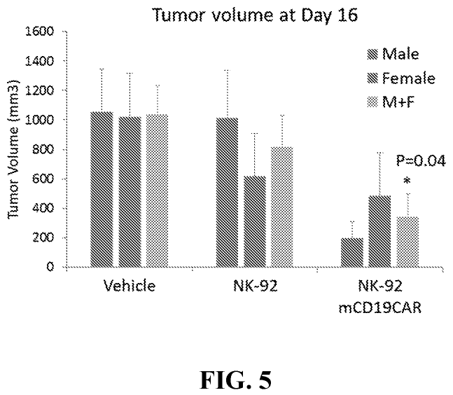

Tumor Growth. Tumor growth was assessed three times each week (3×/week) over the course of the study by digital caliper. For the male animals, tumors treated with parental NK-92 cells (Group 3) displayed similar steady growth kinetics to tumors treated with vehicle (Group 1), with mean tumor volumes exceeding 1000 mm³ by Day 20. In contrast, single A20 tumors of animals treated with mCD19-CAR-NK-92 cells (Group 5) displayed observably inhibited growth kinetics compared to control, with mean tumor volumes not exceeding 1000 mm³ through the full course of the study. For the female animals, animals bearing single tumors treated with either parental NK-92 cells (Group 4) or mCD19-CAR-NK-92 cells (Group 6) displayed inhibited growth kinetics compared to control; indeed mean tumor volumes of Group 6 animals did not exceed 1000 mm³ through the full course of the study. FIG. 5 shows average tumor volumes at Day 16. These results show that within 16 days of treatment, a statistically significant reduction in tumor volumes combining male and female mice are observed.

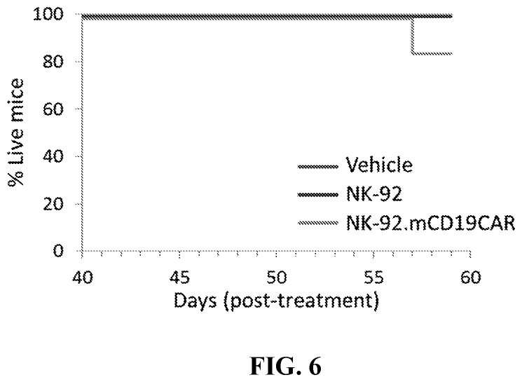

Tumor Re-challenge. To test whether functional immunological memory might persist in animals previously exposed to A20 tumors, animals in Groups 1-6 showing a complete response (i.e. elimination of s.c. tumor mass) were re-challenged with a second s.c. injection of 2.5×10⁶ A20 cells into the right flank on Day 30. The tumor take rate data from the rechallenge portion of the study are summarized in Table 2. FIG. 6 shows all tumor-free mice were re-challenged by a subcutaneous injection of A20 cells in the contralateral flank. All mice remained tumor-free and survived until day 60 post-treatment, except one.

TABLE 2
Re-challenge of tumor free mice at day 30.

| Group (M + F) | Tumor-free at Day 30 | Tumor-free at Day 60 |
|---|---|---|
| Vehicle Control | 1/9 | 1/1 |
| NK-92 Parental Cells | 1/10 | 1/1 |
| NK-92.mCD19CAR | 6/10 | 5/6 |

6/10 mice in the mCD19CAR-treated arm appeared tumor-free by day 30 post-treatment, compared to 1/10 mice in NK-92 treated arm and 1/9 in vehicle treated arm.

### Conclusions

Animal gender did not significantly impact rates of tumor seeding/rejection of syngeneic Balb/c A20 lymphoma cells.

Treatment with mCD19-CAR NK-92 cells provided a statistically significant survival advantage to male mice bearing a single subcutaneous A20 tumor (p=0.0415).

A trend of enhanced survival was also observed for mCD-19-CAR NK-92 treated females bearing single A20 tumors, however this difference fell short of statistical significance (p=0.085).

No statistically significant differences in cumulative weight change were observed when comparing parental or targeted NK-92 treated groups (Group 3-6) to vehicle controls for male or female mice.

For male animals, single A20 tumors treated with mCD19-CAR-NK-92 cells (Group 5) displayed inhibited growth kinetics compared to vehicle treated tumors (Group 1). Cumulative differences in these tumor growth kinetics approached statistical significance (p=0.055).

For female animals, single A20 tumors treated with parental NK-92 cells (Group 4) or mCD19-CAR-NK-92 cells (Group 6) displayed inhibited growth kinetics compared to control. However, no statistically significant differences were detected when comparing cumulative tumor volume of any group to that of vehicle treated controls.

### Example 5

### CD19 CAR NK-92 Cells (CD19 taNK) Induce Complete Remissions in a Highly Aggressive Murine Lymphoma Model (L1210) with Effective Protection Against Re-Challenge

The L1210 malignant lymphoma cell line, derived from DBA/2 mice, is notable both for its short doubling time of 8-10 hours and for its long history of successful use by the NCI in the identification of effective clinical treatments for hematological malignancies. In previous studies we have demonstrated the effectiveness of intra-tumor injection using clinical grade NK-92 (aNK) cells expressing a CAR against the murine CD19 positive A20 lymphoma cell line. This example demonstrates a durable vaccine like effect can be elicited even against this aggressive lymphoma and without the aid of additional checkpoint inhibitors.

### Methods:

Mice used in this Example were DBA/2J male mice, 6-8 weeks of age. All mice were injected with tumor cells on Day PR0. 8 days after tumor cell implantation, when mean tumor volumes were measured to be between 50 and 150 mm³, thirty (30) animals bearing tumors of ˜90 mm³ were selected for enrollment in the study, and these animals were randomized into three (3) groups consisting of ten (10) animals each, with animals in each group bearing tumors of similar mean volume and volume range. Randomization day was considered Day 0 of the study, and treatments were commenced on this day.

### Experimental Design

Sixty (60) male DBA/2J mice aged 6-8 weeks (Jackson Laboratories) were sourced for the study, with thirty (30) of these animals ultimately enrolled following randomization on Day 0. All animals were housed under standard environmental conditions, and were maintained on LabDiet 5053 irradiated rodent chow with sterile water provided ad libitum. On arrival, animals were identified by ear punch and housed in cages of ten (10), and acclimated in place for three days prior to commencement of the study. Following acclimation, the injection area of each mouse was shaved and cleaned with a sterile EtOH swab. On Day PR0 (pre-randomization Day 0), animals were anesthetized with isoflurane for tumor cell injection. All animals were injected with 2×105 L1210-Luc tumor cells subcutaneously (s.c.) into the right flank in a volume of 0.1 mL serum-free DMEM on Day PR0. Beginning on Day PR 7, all animals had tumors measured daily by digital caliper. On Day PR8 (8 days after tumor cell implantation), when tumor volumes were measured at a mean of ˜90 mm3, thirty (30) animals bearing tumors nearest to 90 mm3 were selected for enrollment in the study, and these animals were randomized into three (3) groups consisting of ten (10) animals each from the randomized sample. Randomization day was considered Day 0 of the study, and treatments were commenced on this day.

Animals in Group 1 were administered vehicle (serum free DMEM) as an intratumoral (i.t.) injection of 50 μl. Animals in Group 2 were administered 2×106 NK-92.C cells i.t. in a volume of 50 μl. Animals in Group 3 were administered 2×106 mCD19-CAR-NK-92 cells i.t. in a volume of 50 μl. Identical treatments were administered on Days 0, 2 and 4 of the study. Additional details of intratumoral administration are provided in Methods, below.

Animals were weighed and monitored for general health daily and following randomization, tumors were measured by digital caliper three times each week (3×/week). On Day 30, any animal not bearing a tumor (completely responding to treatment) received an intraperitoneal (i.p.) injection of 150 mg/kg D-luciferin and were imaged by Lumina Series III In Vivo Imaging System (IVIS; PerkinElmer). Additionally on Day 30, completely responding animals were administered a rechallenge tumor cell inoculation of 2×10⁵ L1210-Luc tumor cells subcutaneously (s.c.) into the left flank in a volume of 0.1 mL serum-free DMEM. All animals continued to be weighed and monitored daily and tumor measurements were continued on the 3×/week schedule through Day 60. On Day 60, any animal without a palpable tumor was imaged by IVIS a final time; no collections were scheduled. The details of the study design and group assignments are shown in Table 3 below.

TABLE 3
Study Design

| | | | | | | | Rechallenge | |
|---|---|---|---|---|---|---|---|---|
| | | | | | | | Day 30 | |
| | | | | | | | # Tumor cells | |
| | | | | | | | injected | |
| | | | | | | | s. c. left flank | |
| | | Tumor | | | | | 100 u1 vol | |
| | # Animals | cells | | | | IVIS | Completely | |
| | DBA2/J | # injected | Treatment/ | | | (animals | Responding | |
| | 6-8weeks | s.c. right | # cells | | | w/no | animals only; | |
| | 60 injected | flank | injected (i.t.) | Treatment | Caliper | tumor | Animals w/o | |
| Group | 30 enrolled | 100 u1 vol. | 50 u1 vol | Schedule | Measure | only) | palpable tumor. | Endpoints |
| 1 | 10/male | L1210- | Vehicle | Day 0 | Pre- | Day 30 | L1210-Luc | Day 60 |
| | | Luc | | Day 2 | Randomization | Day 60 | 2.0 × 10⁵ | tumor vol. ≥2500 mm³ |
| | | 2.0 × 10⁵ | | Day 4 | Daily from Day 7 | | | weight loss ≥30% |
| | | | | | Randomized | | | tumor ulcerated |
| | | | | | 3x/week | | | moribund |
| 2 | 10/male | L1210- | NK-92 | Day 0 | Pre- | Day 30 | L1210-Luc | Day 60 |
| | | Luc | parental | Day 2 | Randomization | Day 60 | 2.0 × 10⁵ | tumor vol. ≥2500 mm³ |
| | | 2.0 × 10⁵ | (NK-92.C) | Day 4 | Daily from Day 7 | | | weight loss ≥30% |
| | | | 2 × 10⁶ | | Randomized | | | tumor ulcerated |
| | | | | | 3x/week | | | moribund |
| 3 | 10/male | L1210- | mCD19- | Day 0 | Pre- | Day 30 | L1210-Luc | Day 60 |
| | | Luc | CAR NK-92 | Day 2 | Randomization | Day 60 | 2.0 × 10⁵ | tumor vol. ≥2500 mm³ |
| | | 2.0 × 10⁵ | 2 × 10⁶ | Day 4 | Daily from Day 7 | | | weight loss ≥30% |
| | | | | | Randomized | | | tumor ulcerated |
| | | | | | 3x/week | | | moribund |

The vehicle was 100 μl serum-free DMEM.

### Experimental Procedures

### Cell Culture/Inoculation

L1210-Luc cells were grown under tissue-culture conditions in DMEM supplemented with 10% Horse Serum. The area around the subcutaneous injection sites was shaved prior to injection of tumor cells. On Day PR0, cells were counted by hemocytometer/trypan-blue exclusion and/or MACSquant cytometer. Injection sites were cleaned with sterile ethanol immediately prior to tumor cell inoculations. Animals were administered tumor cells via s.c. injection of 0.1 mL volume to the right flank as indicated in Table 3. The vehicle for all tumor cell injections was 100 μl serum-free DMEM culture media.

### Clinical Assessment/Caliper Measurements

Animals were monitored for survival, weight, and general health on a daily basis. Tumor volumes were assessed by digital caliper daily beginning on Day 7 prior to randomization, and 3×/week post-randomization through study completion. For comparative analysis , tumor volumes were approximated by the formula V=(L*W2)/2.

### Intratumoral Injections

Animals were injected intratumorally (i.t.) with 2×10⁶ test cells in 50 μl volume on Days 0, 2 and 4 as indicated in Table 3. Briefly, cells were delivered by 25 G needle, inserted so that the tip was at the approximate center of the tumor mass. The cells were slowly released into the tumor, and the needle was held in place for a minimum of 30 seconds following dose to allow for the volume to be absorbed into the tumor. The needle was slowly withdrawn as the tumor was squeezed between forceps at the entry site to prevent leakage of the dose from the entry. The forceps was held in place following needle exit for an additional 30 seconds.

### Rechallenge

On Day 30, completely responding animals with no palpable tumor were imaged by IVIS, and received a rechallenge injection of 2×10⁵ L1210-Luc tumor cells. All animals remained on study and continued to be weighed and monitored daily and tumors assessed 3×/week through Day 60. On Day 60 animals without palpable tumors were imaged by IVIS one final time.

### Results and Conclusion:

A Kaplan-Meier survival curve reached significance by both Mantel-Cox test (p=0.0081) and Gehan-Breslow-Wilcoxon test (P=0.0089), with significant inhibition of tumor growth in the treatment cohorts and three durable responses (2 complete with taNK, 1 partial in the aNK cohort). Of the responders, all three showed a vaccine-like effect in their capacity to completely clear a re-challenge dosing of L1210 into the contralateral flank, as compared to 5 naïve controls which all displayed rapid tumor take and lethal outgrowth. To our knowledge, no similar vaccine effect has been described for CAR-T based therapies.

### Example 6

### This Example Demonstrates that Treatment of Mice Having A20 Tumors with mCD19-CAR-NK-92 Cells Increased Survival, and Mice that Completely Responded to Treatment Rejected A20 Tumor Allografts when Re-Challenged

Experimental Design

Part A

Forty (40) 5-7 week old BALB/c mice (20 males and 20 females) were sourced Taconic Biosciences to serve Part A. On pre-randomization (PR) Day 0, animals were injected subcutaneously (s.c.) into the left flank with 2.5×10⁶ A20 murine lymphoma cells in 100 μL volume of serum free media. Beginning on Day PR7, tumors were measured daily. Ten (10) days after tumor cell implantation (Day PR10; Day 0), mice were randomized into treatment groups, such that each group contained animals bearing tumors of similar volume and range. The day of randomization was considered Day 0 of the study. Tumors were measured three times each week (3×/week) by digital caliper to monitor tumor growth until completion of Part A on Day 26.

On Day 0, Day 3, and Day 5, mice were injected intratumorally (i.t.) with test cells or vehicle in 50 μl volume of serum free media into the tumor mass of each animal according to pre-established i.t. procedure (see Experimental Procedures). Briefly, animals were administered vehicle only or were administered 5×106 mCD19-CAR-NK-92. On Day 26, animals that did not develop a tumor of volume >40 mm³ were unenrolled from the study and euthanized by CO2 asphyxiation; enrolled animals that displayed a complete response to treatment (CR; tumors >40 mm³ regressing so as to be undetectable (0 mm³) over multiple days without relapse prior to Day 26) were enrolled in Part B.

Part B

Part B began on Day 26. Animals from Part A without tumors were enrolled in Part B, along with twelve (12) naïve animals (6 males and 6 females). All Part B animals were administered 2.5×10⁶ A20 cells into the right flank. Tumors were measured 2 times/week. Animals were euthanized on Day 57.

Results

Part A—Animal Survival

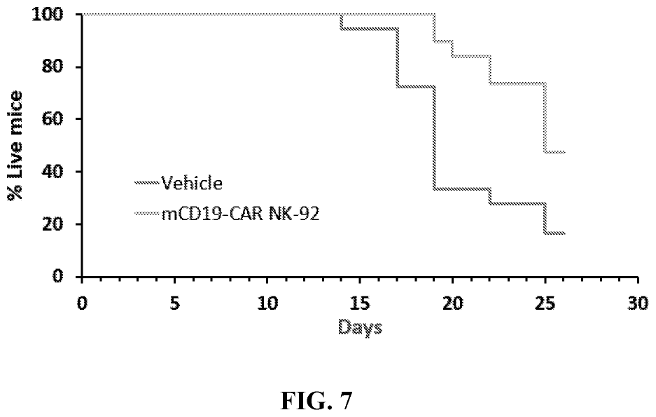

Animals were monitored for general health and survival daily. Animals requiring euthanasia according to animal health and welfare thresholds, including loss of greater than 30% of their initial body weight, tumors exceeding 1500 mm³, inability to obtain food/water or found moribund were included for survival analysis. Animals requiring euthanasia due to ulcerated tumors were not included in survival analysis. In this study, all animals considered in survival analysis were euthanized due to tumor burden exceeding 1500 mm³. As a subcutaneous tumor burden threshold represents an arbitrary cut-off point, the analysis of “survival” in this case must be considered only as an indicator of relative tumor growth. Cumulative survival over time for all animals considered is displayed in FIG. 7.

Of control animals administered vehicle intratumorally (i.t.) on Days 1, 3, and 5: 0 of 15 animals (0%) survived to Part A completion on Day 26. Survival through Day 26 was increased for animals for all animals receiving treatment: 9 out 18 (50%) animals administered 5M mCD19-CAR-NK92 cells. All groups were intercompared by log-rank (Mantel-Cox) test. Compared to animals administered vehicle, a statistically significant enhancement of survival was observed for animals administered 5M mCD19-CAR-NK92 cells (p=<0.0001). These results suggest that all treatments improved survival through Day 26 compared to treatment with vehicle.

Part B—Tumor Re-Challenge of Complete Responders

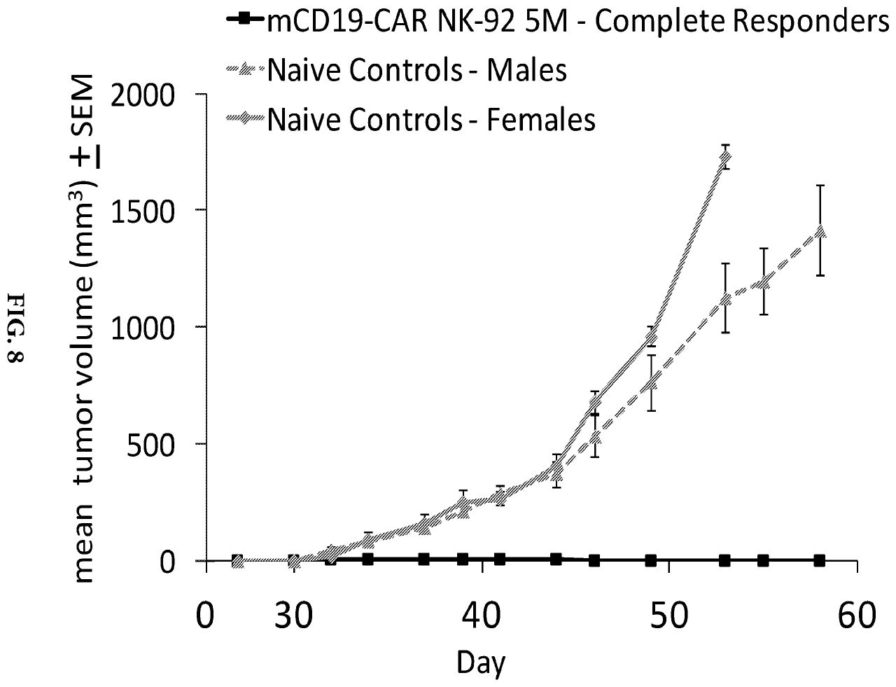

Animals that completely responded to treatment (bearing a tumor >40 mm³ that responded to treatment over the course of Day 0-26 (Part A) such that the tumor volume measured 0.00 mm³ through Day 26 without regrowth or relapse) were re-challenged with a second subcutaneous inoculation into the flank (opposite side from the first graft), with 2.5×10⁶ A20 tumor cells in 0.1 mL serum free RPMI-1640 media on Day 27; the rechallenge portion of the study was designated as Part B. An additional twelve animals were enrolled into Part B the study to serve as naïve controls; six (6) male and six (6) female age-matched BALB/c mice sourced at the same time and vendor as Part A mice were administered 2.5×10⁶ A20 tumor cells on Day 27. Tumors were measured 3 times/week for all animals through Day 57. Mean tumor volumes+SEM of each Part A treatment group and naïve controls are shown in FIG. 8. Tumors derived from cell inoculations into naïve animals grew steadily as expected; whereas re-challenge tumor cell inoculations into complete responder animals did not produce viable tumors (>40 mm³).

In summary, the data presented in this Example indicates that, in contrast to naïve mice, previously treated mice that completely responded to treatment were able to reject A20 tumor allografts applied as re-challenge regardless of the treatment, and suggests that that these animals developed a memory response to tumor antigens.

### Example 7

### This Example Demonstrates that Treatment of Mice Having L1210 Tumors with CD19-CAR-NK-92 Cells Increased Survival, and Mice that Completely Responded to Treatment Rejected L1210 Tumor Allografts when Re-Challenged

Experimental Design

Thirty (30) male DBA/2J mice aged 6-8 weeks (Jackson Laboratories) were enrolled following randomization on Day 0. All animals were housed under standard environmental conditions and maintained on LabDiet 5053 irradiated rodent chow and sterile water provided ad libitum. On arrival, animals were identified by ear punch and housed in cages of ten (10) and acclimated in place for a minimum of three days prior to commencement of the study. Following acclimation, the injection area of each mouse was shaved and cleaned with sterile EtOH swab. On Day PR0 (pre-randomization Day 0), animals were anesthetized with isoflurane for tumor cell injection. All animals were injected with 2×10⁵ L1210-Luc tumor cells subcutaneously (s.c.) into the right flank in a volume of 0.1 mL serum-free DMEM on Day PR0. Beginning on Day PR 7, all animals had tumors measured daily by digital caliper. On ˜Day PR7 when tumor volumes were measured at ˜50-150 mm³, and mean tumor volume was measured at ˜100 mm³, the twenty (20) animals bearing tumors nearest to ˜100 mm³ were selected for enrollment in the study; these animals were randomized into two (2) groups consisting of ten (10) animals each. Randomization day was considered Day 0 of the study, and administration of treatments commenced on this day. Animals not enrolled on study were immediately euthanized by CO2 overdose. Animals in Group 1 were administered vehicle (serum free DMEM) as an intratumoral (i.t.) injection of 50 μl. Animals in Group 2 were administered 2×106 mCD19-CAR-aNK cells i.t. in a volume of 50 μl. Identical treatments were administered on Days 0, 2 and 4 of the study.

Animals were weighed and monitored for general health daily. Following randomization, tumors were measured by digital caliper three times each week (3×/week). Any animal bearing a tumor >2500 mm³ or a tumor that has ulcerated; that lost >30% of its initial body weight (on Day 0); or was found moribund, distressed or paralyzed was euthanized by CO2 overdose with cause of death/sacrifice noted. On Day 30, completely responding animals and five (5) naïve additional male DBA/2J mice aged ˜10 weeks (Jackson Laboratories; Barrier) comprising Group 4 were administered a rechallenge tumor cell inoculation of 2×105 L1210-Luc tumor cells subcutaneously (s.c.) into the left flank in a volume of 0.1 mL serum-free DMEM. All animals continued to be weighed and monitored daily and tumor measurements continued 3×/week through Day 60.

Results

Animal Survival to Welfare Thresholds—Initial Tumor Challenge

Animals were monitored for survival daily. Animals requiring euthanasia according to animal health and welfare thresholds, including loss of greater than 30% of their initial body weight, tumors exceeding 2500 mm³, inability to obtain food/water, or found moribund, were included for survival analysis. Animals requiring euthanasia due to ulcerated tumors were not included in survival analysis.

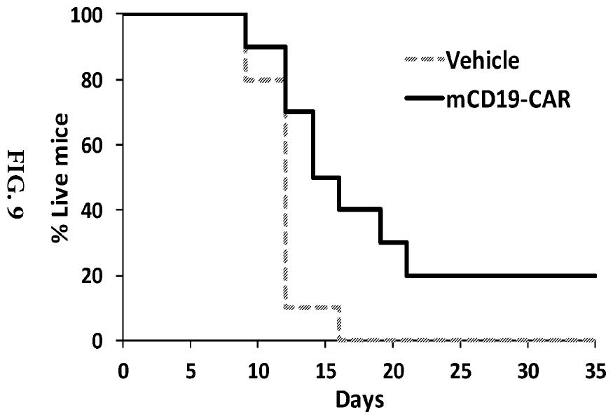

Cumulative survival to animal welfare thresholds over time is displayed in FIG. 9. L1210 is an extremely fast-growing, aggressive tumor cell line and 0% of vehicle treated control animals survived further than twenty-three (23) days post tumor challenge. In contrast, treatment with CD19-CAR-aNK cells enhanced survival compared to treatment with vehicle. Indeed, 25% (2/8) of animals treated with CD19-CAR-aNK cells survived through study completion at Day 61 through tumor graft challenge.

The statistical significance of the observed survival enhancements provided by the test treatments was assessed by Log-rank (Mantel-Cox) and Gehan-Breslow Wilcoxon tests. Treatment with mCD19-CAR-aNK cells produced a statistically significant enhancement of survival, (p=0.05 (Mantel-Cox); p=0.04 (Gehan-Breslow-Wilcoxon). These results indicate that treatment with CD19-CAR-aNK produced statistically significant improvement of survival to welfare threshold compared to vehicle in this preclinical subcutaneous model of murine lymphocytic leukemia.

Tumor Re-Challenge of Complete Responders

On Day 33, the two (2) complete responding animals from Group 2, along with five (5) age-matched naïve animals were challenged/rechallenged with a second inoculum of 2×10⁵ L1210-Luc cells, injected into the opposite (left) flank (primary tumor was seeded into the right flank). Animals were monitored for survival daily. Animals requiring euthanasia according to animal health and welfare thresholds, including loss of greater than 30% of their initial body weight, tumors exceeding 2500 mm³, inability to obtain food/water, or found moribund, were included for survival analysis. Animals requiring euthanasia due to ulcerated tumors were not included in survival analysis.

All (5 of 5) survival analysis eligible naïve animals required euthanization due to tumor volume by Day 52; in contrast, all completely responding animals previously treated with 2M CD19-CAR-aNK (N=2) cells survived through study completion (Day 62). The statistical significance of the observed survival enhancement provided by the test treatments was assessed by Log-rank (Mantel-Cox) and Gehan-Breslow Wilcoxon tests, however the enhancement in survival was not statistically distinguishable, most likely to due to small sample sizes.

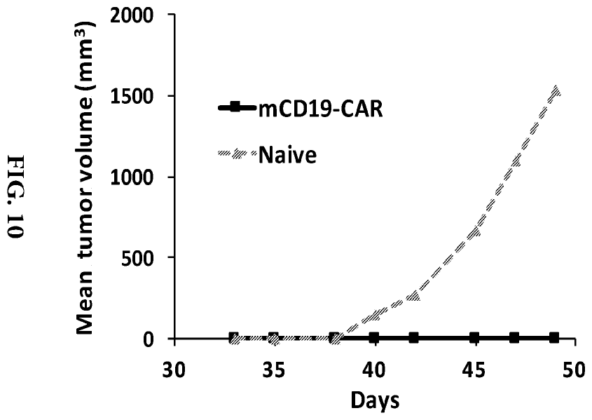

Tumors continued to be measured three times each week (3×/week) during the rechallenge phase. The mean tumor volume+SEM for each group from administration of challenge/rechallenge L1210-Luc cells to 0% control group survival (Day 52) are displayed in FIG. 10.

Tumors of naïve animals were first detectable about seven days after administration (on study Day 40) and increased steadily and rapidly. In contrast, no tumors were detected following rechallenge into completely responding animals previously treated with 2M CD19-CAR-aNK cells at any point over the full course of the rechallenge phase (Day 33-61).

The data provided in this example suggest that completely responding animals previously treated with 2M CD19-CAR-aNK cells may have developed an effective immune response to L1210 tumor cells.

All patents, patent applications, publications, and sequence accession numbers (e.g., Genbank accession numbers) described herein are incorporated by reference herein.

Informal Sequence Listing

SEQ ID NO: 1. Polynucleotide Encoding the Low Affinity Immunoglobulin Gamma Fc Region
Receptor III-A (Precursor) (Encodes phenylalanine at position 158)
atgtggcagc tgctcctccc aactgctctg ctacttctag tttcagctgg catgcgg act gaagatctcc caaaggctgt ggtgttcctg
gagcctcaat ggtacagggt gctcgagaag gacagtgtga ctctgaagtg ccagggagcc tactcccctg aggacaattc
cacacagtgg tttcacaatg agagcctcat ctcaagccag gcctcgagct acttcattga cgctgccaca gtcgacgaca
gtggagagta caggtgccag acaaacctct ccaccctcag tgacccggtg cagctagaag tccatatcgg ctggctgttg
ctccaggccc ctcggtgggt gttcaaggag gaagacccta ttcacctgag gtgtcacagc tggaagaaca ctgctctgca
taaggtcaca tatttacaga atggcaaagg caggaagtat tttcatcata attctgactt ctacattcca aaagccacac tcaaagacag
cggctcctac ttctgcaggg ggctttttgg gagtaaaaat gtgtcttcag agactgtgaa catcaccatc actcaaggtt tggcagtgtc
aaccatctca tcattctttc cacctgggta ccaagtctct ttctgcttgg tgatggtact ccifittgca gtggacacag gactatattt
ctctgtgaag acaaacattc gaagctcaac aagagactgg aaggaccata aatttaaatg gagaaaggac cctcaagaca aatga
SEQ ID NO: 2. CD19-CAR DNA sequence (murine)
CCCGGGAATT CGCCACCATG GACTGGATCT GGCGGATCCT GTTCCTCGTG GGAGCCGCCA
CAGGCGCCCA TTCTGCCCAG CCCGCCGACA TCCAGATGAC CCAGACCACC AGCAGCCTGA
GCGCCAGCCT GGGCGACAGA GTGACCATCA GCTGCCGGGC CAGCCAGGAC ATCAGCAAGT
ACCTGAACTG GTATCAGCAG AAACCCGACG GCACCGTGAA GCTGCTGATC TACCACACCA
GCCGGCTGCA CAGCGGCGTG CCCAGCAGAT TTTCTGGCAG CGGCAGCGGC ACCGACTACA
GCCTGACCAT CTCCAACCTG GAACAGGAAG ATATCGCTAC CTACTTCTGT CAGCAAGGCA
ACACCCTGCC CTACACCTTC GGCGGAGGCA CCAAGCTGGA ACTGAAGAGA GGCGGCGGAG
GCTCTGGTGG AGGCGGATCT GGGGGCGGAG GAAGTGGCGG GGGAGGATCT GAAGTGCAGC
TGCAGCAGAG CGGCCCTGGC CTGGTGGCCC CTAGCCAGAG CCTGTCCGTG ACCTGTACCG
TGTCCGGCGT GTCCCTGCCC GACTACGGCG TGTCCTGGAT CCGGCAGCCC CCCAGAAAGG
GCCTGGAATG GCTGGGCGTG ATCTGGGGCA GCGAGACAAC CTACTACAAC AGCGCCCTGA
AGTCCCGGCT GACCATCATC AAGGACAACA GCAAGAGCCA GGTGTTCCTG AAGATGAACA
GCCTGCAGAC CGACGACACC GCCATCTACT ACTGCGCCAA GCACTACTAC TACGGCGGCA
GCTACGCCAT GGACTACTGG GGCCAGGGCA CCACCGTGAC CGTGTCCAGC GCCCTGTCCA
ACAGCATCAT GTACTTCAGC CACTTCGTGC CCGTGTTTCT GCCCGCCAAG CCCACCACCA
CCCCTGCCCC TAGACCTCCC ACCCCAGCCC CAACAATCGC CAGCCAGCCT CTGTCCCTGC
GGCCCGAAGC TAGCAGACCT GCTGCCGGCG GAGCCGTGCA CACCAGAGGC CTGGACCCCA
AGCTGTGCTA CCTGCTGGAC GGCATCCTGT TCATCTATGG CGTGATCCTG ACCGCCCTGT
TCCTGAGAGT GAAGTTCAGC AGAAGCGCCG ACGCCCCTGC CTACCAGCAG GGCCAGAACC
AGCTGTACAA CGAGCTGAAC CTGGGCAGAC GGGAAGAGTA CGACGTGCTG GACAAGCGGA
GAGGCAGGGA CCCCGAGATG GGCGGCAAGC CCAGACGGAA GAACCCCCAG GAAGGCCTGT
ATAACGAACT GCAGAAAGAC AAGATGGCCG AGGCCTACAG CGAGATCGGC ATGAAGGGCG
AGCGGCGGAG GGGCAAGGGC CACGATGGAC TGTACCAGGG CCTGAGCACC GCCACCAAGG
ACACCTACGA CGCCCTGCAC ATGCAGGCCC TGCCCCCCAG ATGACAGCCA GGGCATTTCT
CCCTCGAGCG GCCGC
SEQ ID NO: 3. CD19-CAR amino acids sequence (murine)
MDWIWRILFL VGAATGAHSA QPADIQMTQT TSSLSASLGD RVTISCRASQ DISKYLNWYQ
QKPDGTVKLL IYHTSRLHSG VPSRFSGSGS GTDYSLTISN LEQEDIATYF CQQGNTLPYT
FGGGTKLELK RGGGGSGGGG SGGGGSGGGG SEVQLQQSGP GLVAPSQSLS VTCTVSGVSL
PDYGVSWIRQ PPRKGLEWLG VIWGSETTYY NSALKSRLTI IKDNSKSQVF LKMNSLQTDD
TAIYYCAKHY YYGGSYAMDY WGQGTTVTVS SALSNSIMYF SHFVPVFLPA KPTTTPAPRP
PTPAPTIASQ PLSLRPEASR PAAGGAVHTR GLDPKLCYLL DGILFIYGVI LTALFLRVKF
SRSADAPAYQ QGQNQLYNEL NLGRREEYDV LDKRRGRDPE MGGKPRRKNP QEGLYNELQK
DKMAEAYSEI GMKGERRRGK GHDGLYQGLS TATKDTYDAL HMQALPPR
SEQ ID NO: 4. Codon-optimized CD19 scFv-DNA sequence:
ATGGACTGGATCTGGCGGATtCTGTTTCTCGTGGGAGCTGCCACAGGCGCTCATTCTGCTCAGC
CTGCCGATATCCAGATGACCCAGACAACAAGCAGCCTGAGCGCCTCTCTGGGCGATAGAGTGAC
AATCAGCTGCAGAGCCAGCCAGGACATCAGCAAGTACCTGAACTGGTATCAGCAGAAACCCGAC
GGCACCGTGAAGCTGCTGATCTACCACACAAGCAGACTGCACAGCGGCGTGCCAAGCAGATTTT
CTGGCAGCGGCAGCGGCACCGATTACAGCCTGACCATCAGCAACCTGGAACAGGAAGATATCG
CTACCTACTTCTGTCAGCAGGGCAACACCCTGCCTTACACCTTTGGCGGCGGAACAAAGCTGGA
ACTGAAAAGAGGCGGCGGAGGAAGCGGAGGCGGAGGATCTGGGGGCGGAGGCTCTGGCGGA
GGGGGATCTGAAGTGCAGCTGCAGCAGTCTGGACCTGGACTGGTGGCTCCTTCTCAGTCCCTG
TCTGTGACCTGTACAGTGTCTGGCGTGTCCCTGCCTGATTACGGCGTGTCCTGGATCAGACAGC
CTCCCAGAAAAGGCCTGGAATGGCTGGGAGTGATCTGGGGCAGCGAGACAACCTACTACAACA
GCGCCCTGAAGTCCCGGCTGACCATCATCAAGGACAACAGCAAGAGCCAGGTGTTCCTGAAGAT
GAACAGCCTGCAGACCGACGACACCGCCATCTACTACTGCGCCAAGCACTACTACTACGGCGG
CAGCTACGCCATGGATTATTGGGGCCAGGGCACCACCGTGACAGTGTCATCT
ATG = start codon
SEQ ID NO: 5. CD19 scFv- Protein sequence:
MDWIWRILFLVGAATGAHSAQPADIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLELKRGGGG
SGGGGSGGGGSGGGGSEVQLQQSGPGLVAPSQSLSVTCTVSGVSLPDYGVSWIRQPPRKGLEWL
GVIWGSETTYYNSALKSRLTIIKDNSKSQVFLKMNSLQTDDTAIYYCAKHYYYGGSYAMDYWGQGTT
VTVSS
SEQ ID NO: 6. Codon-optimized CD20 scFv-DNA sequence:
ATGGACTGGATCTGGCGCATCCTCTTCCTCGTCGGCGCTGCTACCGGCGCTCATTCGGCCCAG
CCGGCCATGGCGCAAGTAAAACTCCAAGAATCTGGGGCGGAGCTGGTGAAACCGGGGGCGTCT
GTGAAGATGAGCTGTAAAGCATCAGGCTACACCTTCACCTCCTATAATATGCACTGGGTGAAACA
AACACCCGGACAGGGCCTCGAATGGATTGGTGCCATCTATCCTGGAAATGGTGATACCTCATAT
AATCAGAAGTTTAAGGGCAAGGCTACGCTTACTGCGGATAAAAGCTCTTCCACTGCTTACATGCA
ACTGAGCAGTCTCACTTCAGAGGACTCAGCCGATTATTATTGTGCCCGCAGCAACTACTATGGTA
GTTCATACTGGTTTTTCGACGTTTGGGGGCAAGGTACCACCGTCACGGTTTCTTCTGGTGGGGG
CGGAAGCGGGGGTGGAGGATCTGGGGGCGGTGGTTCAGACATTGAACTCACCCAGAGCCCTAC
TATTCTGAGCGCGTCTCCAGGTGAAAAAGTTACGATGACGTGCAGAGCATCAAGTAGTGTGAATT
ATATGGATTGGTATCAAAAGAAGCCAGGCTCATCCCCAAAACCGTGGATCTATGCAACTAGCAAC
CTCGCGTCAGGGGTGCCAGCAAGGTTTTCCGGAAGTGGTTCTGGCACATCTTATAGTCTCACCA
TTTCCCGAGTGGAGGCTGAGGATGCGGCCACTTATTACTGCCAGCAATGGTCATTCAATCCCCC
AACATTTGGTGGCGGAACAAAACTCGAAATTAAACGG
ATG = start codon
SEQ ID NO: 7. CD20 scFv-Protein sequence:
MDWIWRILFLVGAATGAHSAQPAMAQVKLQESGAELVKPGASVKMSCKASGYTFTSYNMHWVKQT
PGQGLEWIGAIYPGNGDTSYNQKFKGKATLTADKSSSTAYMQLSSLTSEDSADYYCARSNYYGSSY
WFFDVWGQGTTVTVSSGGGGSGGGGSGGGGSDIELTQSPTILSASPGEKVTMTCRASSSVNYMD
WYQKKPGSSPKPWIYATSNLASGVPARFSGSGSGTSYSLTISRVEAEDAATYYCQQWSFNPPTFGG
GTKLEIKR
SEQ ID NO: 8. Codon-optimized CD33 scfV-DNA sequence:
ATGGACTGGATCTGGCGCATCCTCTTCCTCGTCGGCGCTGCTACCGGCGCTCATTCGGCCCAG
CCGGCCGACATTCAAATGACTCAGTCCCCTTCCAGCTTGTCAGCCTCAGTAGGGGACCGGGTCA
CGATCACCTGTCGAGCGTCTGAGTCAGTGGATAACTACGGGATTTCTTTCATGAACTGGTTCCAG
CAGAAGCCCGGCAAAGCTCCTAAGCTCCTTATATATGCAGCCTCAAATCAGGGGAGCGGTGTTC
CTAGTCGCTTCAGTGGAAGCGGTAGCGGTACGGACTTTACGTTGACGATAAGTAGCCTTCAGCC
AGATGACTTTGCCACTTATTATTGTCAGCAGTCTAAGGAAGTTCCTTGGACGTTTGGCCAAGGAA
CGAAGGTCGAAATCAAAGGGGGAGGGGGCTCAGGAGGGGGCGGCAGTGGTGGTGGAGGCTCT
CAAGTCCAACTCGTACAGTCTGGCGCGGAGGTTAAAAAGCCGGGAAGCTCCGTGAAAGTATCCT
GTAAGGCAAGCGGATACACCTTTACCGATTATAACATGCACTGGGTTAGGCAGGCGCCCGGCCA
AGGTCTGGAATGGATCGGTTATATTTATCCATACAACGGTGGTACCGGCTATAATCAGAAGTTTA
AGAGTAAGGCTACTATTACAGCGGATGAGTCAACCAATACTGCATACATGGAGCTCTCCTCACTC
AGGAGCGAAGATACCGCAGTGTATTACTGTGCCCGAGGGAGACCAGCCATGGACTACTGGGGT
CAGGGTACCCTTGTGACAGTATCTAGC
ATG = start codon
SEQ ID NO: 9. CD33 scfV-Protein sequence:
MDWIWRILFLVGAATGAHSAQPADIQMTQSPSSLSASVGDRVTITCRASESVDNYGISFMNWFQQKP
GKAPKLLIYAASNQGSGVPSRFSGSGSGTDFTLTISSLQPDDFATYYCQQSKEVPWTFGQGTKVEIK
GGGGSGGGGSGGGGSQVQLVQSGAEVKKPGSSVKVSCKASGYTFTDYNMHWVRQAPGQGLEWI
GYIYPYNGGTGYNQKFKSKATITADESTNTAYMELSSLRSEDTAVYYCARGRPAMDYWGQGTLVTV
SS
SEQ ID NO: 10. Codon-optimized CSPG4 scfV-DNA sequence:
ATGGACTGGATCTGGCGCATCCTCTTCCTCGTCGGCGCTGCTACCGGCGCTCATTCGGCCCAG
CCGGCCGATATCGAGCTCACCCAATCTCCAAAATTCATGTCCACATCAGTAGGAGACAGGGTCA
GCGTCACCTGCAAGGCCAGTCAGAATGTGGATACTAATGTAGCGTGGTATCAACAAAAACCAGG
GCAATCTCCTGAACCACTGCTTTTCTCGGCATCCTACCGTTACACTGGAGTCCCTGATCGCTTCA
CAGGCAGTGGATCTGGGACAGATTTCACTCTCACCATCAGCAATGTGCAGTCTGAAGACTTGGC
AGAGTATTTCTGTCAGCAATATAACAGCTATCCTCTGACGTTCGGTGGCGGCACCAAGCTGGAAA
TCAAACGGGCTGCCGCAGAAGGTGGAGGCGGTTCAGGTGGCGGAGGTTCCGGCGGAGGTGGC
TCTGGCGGTGGCGGATCGGCCATGGCCCAGGTGAAGCTGCAGCAGTCAGGAGGGGGCTTGGT
GCAACCTGGAGGcTCCATGAAACTCTCCTGTGTTGTCTCTGGATTCACTTTCAGTAATTACTGGAT
GAACTGGGTCCGCCAGTCTCCAGAGAAGGGGCTTGAGTGGATTGCAGAAATTAGATTGAAATCC
AATAATTTTGGAAGATATTATGCGGAGTCTGTGAAAGGGAGGTTCACCATCTCAAGAGATGATTC
CAAAAGTAGTGCCTACCTGCAAATGATCAACCTAAGAGCTGAAGATACTGGCATTTATTACTGTA
CCAGTTATGGTAACTACGTTGGGCACTATTTTGACCACTGGGGCCAAGGGACCACGGTCACCGT
ATCGAGT
ATG = start codon
SEQ ID NO: 11. CSPG4 scfV-Protein sequence:
MDWIWRILFLVGAATGAHSAQPADIELTQSPKFMSTSVGDRVSVTCKASQNVDTNVAWYQQKPGQS
PEPLLFSASYRYTGVPDRFTGSGSGTDFTLTISNVQSEDLAEYFCQQYNSYPLTFGGGTKLEIKRAAA
EGGGGSGGGGSGGGGSGGGGSAMAQVKLQQSGGGLVQPGGSMKLSCVVSGFTFSNYWMNWVR
QSPEKGLEWIAEIRLKSNNFGRYYAESVKGRFTISRDDSKSSAYLQMINLRAEDTGIYYCTSYGNYVG
HYFDHWGQGTTVTVSS
SEQ ID NO: 12. Codon-optimized EGFR scFv-DNA sequence:
ATGGACTGGATCTGGCGGATTCTGTTTCTCGTGGGAGCTGCCACAGGCGCTCATTCTGCTCAGC
CTGCCGATATTCTTCTTACTCAATCTCCCGTTATTTTGTCAGTATCCCCAGGTGAGCGAGTCAGCT
TCTCTTGTCGAGCGTCACAATCCATTGGCACCAACATACATTGGTACCAACAGCGCACCAACGG
GTCTCCCCGGCTCTTGATTAAGTACGCATCAGAAAGTATTTCTGGGATACCCAGTAGGTTCTCAG
GGAGCGGGAGTGGCACTGACTTTACCCTGTCCATAAACAGCGTTGAGTCTGAGGACATCGCGGA
CTACTATTGTCAGCAGAACAACAATTGGCCGACCACGTTTGGTGCGGGAACAAAACTTGAACTCA
AAGGCGGCGGAGGAAGCGGAGGCGGAGGATCTGGGGGCGGAGGCTCTGGCGGAGGGGGATC
TCAGGTGCAGCTCAAACAGTCAGGACCTGGCCTCGTTCAGCCAAGCCAATCACTGAGTATAACG
TGCACGGTGAGCGGCTTTAGCCTGACAAACTATGGTGTCCACTGGGTCCGCCAATCTCCTGGAA
AAGGCTTGGAGTGGCTCGGTGTTATCTGGTCCGGTGGTAACACAGACTACAACACGCCATTCAC
CAGTCGCCTTAGTATTAACAAGGACAACTCCAAGTCTCAGGTTTTCTTTAAAATGAACTCTCTGCA
GTCTAATGATACCGCAATTTACTACTGTGCGAGGGCACTCACGTACTATGACTATGAGTTCGCGT
ATTGGGGCCAAGGGACTCTCGTTACTGTCTCAGCG
ATG = start codon
SEQ ID NO: 13. EGFR scFv-Protein sequence:
MDWIWRILFLVGAATGAHSAQPADILLTQSPVILSVSPGERVSFSCRASQSIGTNIHWYQQRTNGSPR
LLIKYASESISGIPSRFSGSGSGTDFTLSINSVESEDIADYYCQQNNNWPTTFGAGTKLELKGGGGSG
GGGSGGGGSGGGGSQVQLKQSGPGLVQPSQSLSITCTVSGFSLTNYGVHWVRQSPGKGLEWLGVI
WSGGNTDYNTPFTSRLSINKDNSKSQVFFKMNSLQSNDTAIYYCARALTYYDYEFAYWGQGTLVTVS
A
SEQ ID NO: 14. Codon-optimized IGF1R scFv-DNA sequence:
ATGGACTGGATCTGGCGGATTCTGTTTCTCGTGGGAGCTGCCACAGGCGCTCATTCTGCTCAGC
CTGCCGATGTTGTAATGACGCAGTCACCCCTGTCACTCCCGGTCACACCCGGAGAACCAGCGTC
AATTAGCTGCCGATCTAGCCAAAGTTTGCTTCATTCCAATGGTTACAATTATCTCGACTGGTACTT
GCAGAAACCCGGCCAATCCCCTCAGCTGCTCATCTACCTTGGGTCTAATAGGGCATCTGGGGTT
CCCGATAGGTTCTCTGGCTCCGGGAGCGGCACCGACTTTACGTTGAAAATCTCTAGGGTTGAGG
CGGAAGACGTAGGCGTTTACTATTGCATGCAGGGGACCCACTGGCCGCTGACCTTCGGCCAGG
GCACCAAGGTTGAAATAAAAGGCGGCGGAGGAAGCGGAGGCGGAGGATCTGGGGGCGGAGGC
TCTGGCGGAGGGGGATCTCAGGTACAGCTCCAGGAATCAGGACCCGGTTTGGTTAAGCCCTCC
GGGACCCTTTCCCTCACGTGTGCAGTCTCAGGTGGGTCAATTAGTTCTTCCAATTGGTGGTCTTG
GGTGCGGCAACCACCTGGTAAAGGTCTCGAGTGGATAGGGGAAATTTATCATAGTGGCTCCACC
AATTATAACCCCTCACTCAAGTCCAGGGTTACGATATCTGTGGACAAAAGTAAAAACCAATTCTCC
CTCAAACTTAGTAGTGTAACAGCGGCAGACACCGCGGTGTACTACTGCGCACGGTGGACAGGC
CGAACTGATGCCTTTGACATTTGGGGACAGGGAACTATGGTGACTGTGTCATCC
ATG = start codon
SEQ ID NO: 15. IGF1R scFv-Protein sequence:
MDWIWRILFLVGAATGAHSAQPADVVMTQSPLSLPVTPGEPASISCRSSQSLLHSNGYNYLDWYLQK
PGQSPQLLIYLGSNRASGVPDRFSGSGSGTDFTLKISRVEAEDVGVYYCMQGTHWPLTFGQGTKVEI
KGGGGSGGGGSGGGGSGGGGSQVQLQESGPGLVKPSGTLSLTCAVSGGSISSSNWWSWVRQPP
GKGLEWIGEIYHSGSTNYNPSLKSRVTISVDKSKNQFSLKLSSVTAADTAVYYCARWTGRTDAFDIW
GQGTMVTVSS
SEQ ID NO: 16. Codon-optimized CD30 scFv-DNA sequence:
ATGGACTGGATCTGGCGGATTCTGTTTCTCGTGGGAGCTGCCACAGGCGCTCATTCTGCTCAGC
CTGCCGATATCCAAATGACTCAATCTCCTAGTTCACTGTCAGCCTCTGTTGGTGATCGCGTGACC
ATTACCTGCCAAGCTAGCCAGGATATTAGCAACTACTTGAACTGGTATCAGCAGAAGCCTGGCAA
AGCCCCAAAGCTGTTGATCTACGATGTAAGTAACTTGGAAACTGGCGTCCCAAGCCGCTTCTCTG
GATCTGGTTCAGGCACCGACTTCACTTTCACTATCAGCAGCCTGCAGCCTGAAGATATCGCAACC
TACTATTGCCAGCAGGTTGCTAATGTTCCTCTGACTTTCGGCCAAGGCACCAAGGTGGAGATCAA
GGGCGGCGGAGGAAGCGGAGGCGGAGGATCTGGGGGCGGAGGCTCTGGCGGAGGGGGATCT
GAAGTTCAGCTTGTAGAATCTGGAGGTGGATTGGTTCAACCTGGTGGCTCTCTTCGCCTGAGTT
GTGCAGCCTCTGGTTTTACTTTCTCTAGTTACTGGATGCATTGGGTTCGTCAGGCTCCTGGGAAA
GGCCTGGAATGGGTTTCAGCTATTAGTTGGAGTGGAGATAGTACTTACTACGCAGACAGTGTGA
AAGGTCGCTTCACCATCAGCCGTGATAATTCTAAGAACACTTTGTACCTGCAAATGAACTCCTTG
CGCGCAGAAGACACGGCTGTGTACTATTGTGCCCGTGATCGCTCTGCGACTTGGTATTATCTGG
GGCTTGGTTTCGATGTATGGGGACAAGGTACCCTGGTAACGGTTTCTAGC
ATG = start codon
SEQ ID NO: 17. CD30 scFv-Protein sequence:
MDWIWRILFLVGAATGAHSAQPADIQMTQSPSSLSASVGDRVTITCQASQDISNYLNWYQQKPGKAP
KLLIYDVSNLETGVPSRFSGSGSGTDFTFTISSLQPEDIATYYCQQVANVPLTFGQGTKVEIKGGGGS
GGGGSGGGGSGGGGSEVQLVESGGGLVQPGGSLRLSCAASGFTFSSYWMHWVRQAPGKGLEWV
SAISWSGDSTYYADSVKGRFTISRDNSKNTLYLQMNSLRAEDTAVYYCARDRSATWYYLGLGFDVW
GQGTLVTVSS
SEQ ID NO: 18. Codon-optimized HER2/neu scFv-DNA sequence:
ATGGACTGGATCTGGCGGATTCTGTTTCTCGTGGGAGCTGCCACAGGCGCTCATTCTGCTCAGC
CTGCCGACATCCAGATGACCCAGAGCCCCAGCAGCCTGAGCGCCAGCGTGGGCGACAGAGTGA
CCATCACCTGCAGAGCCAGCCAGGACGTGAACACCGCCGTGGCCTGGTACCAGCAGAAGCCCG
GCAAGGCCCCCAAGCTGCTGATCTACAGCGCCAGCTTCCTGTACAGCGGCGTGCCCAGCAGAT
TCAGCGGCAGCAGAAGCGGCACCGACTTCACCCTGACCATCAGCAGCCTGCAGCCCGAGGACT
TCGCCACCTACTACTGCCAGCAGCACTACACCACCCCCCCCACCTTCGGCCAGGGCACCAAGG
TGGAGATCAAGTCCTCAGGGGGCGGGGGAAGTGGTGGGGGCGGCAGCGGCGGAGGGGGCTC
AGGAGGAGGCGGATCAGGCGGATCAGAGGTGCAGCTGGTGGAGAGCGGCGGCGGCCTGGTG
CAGCCCGGCGGCAGCCTGAGACTGAGCTGCGCCGCCAGCGGCTTCAACATCAAGGACACCTAC
ATCCACTGGGTGAGACAGGCCCCCGGCAAGGGCCTGGAGTGGGTGGCCAGAATCTACCCCACC
AACGGCTACACCAGATACGCCGACAGCGTGAAGGGCAGATTCACCATCAGCGCCGACACCAGC
AAGAACACCGCCTACCTGCAGATGAACAGCCTGAGAGCCGAGGACACCGCCGTGTACTACTGC
AGCAGATGGGGCGGCGACGGCTTCTACGCCATGGACTACTGGGGCCAGGGCACCCTGGTGAC
CGTGAGCAGC
ATG = start codon
SEQ ID NO: 19. HER2/neu scFv-Protein sequence:
MDWIWRILFLVGAATGAHSAQPADIQMTQSPSSLSASVGDRVTITCRASQDVNTAVAWYQQKPGKA
PKLLIYSASFLYSGVPSRFSGSRSGTDFTLTISSLQPEDFATYYCQQHYTTPPTFGQGTKVEIKSSGG
GGSGGGGSGGGGSGGGGSGGSEVQLVESGGGLVQPGGSLRLSCAASGFNIKDTYIHWVRQAPGK
GLEWVARIYPTNGYTRYADSVKGRFTISADTSKNTAYLQMNSLRAEDTAVYYCSRWGGDGFYAMDY
WGQGTLVTVSS
SEQ ID NO: 20. Codon-optimized GD2 scFv-DNA sequence VL/VH format:
ATGGACTGGATCTGGCGGATTCTGTTTCTCGTGGGAGCTGCCACAGGCGCTCATTCTGCTCAGC
CTGCCAGCATCGTGATGACCCAGACTCCTAAGTTCCTGCTGGTGTCTGCCGGCGACAGAGTGAC
CATCACCTGTAAAGCCAGCCAGAGCGTGTCCAACGACGTGGCCTGGTATCAGCAGAAGCCTGG
ACAGAGCCCCAAGCTGCTGATCTACAGCGCCAGCAACAGATACACCGGCGTGCCCGATAGATTC
ACCGGCTCTGGCTACGGCACCGACTTCACCTTTACCATCAGCACCGTGCAGGCCGAGGATCTG
GCCGTGTACTTCTGCCAGCAAGACTACAGCTCTCTCGGCGGAGGCACCAAGCTGGAAATCAAAG
GCGGCGGAGGAAGCGGAGGCGGAGGATCTGGGGGCGGAGGCTCTGGCGGAGGGGGATCTCA
GGTGCAAGTGAAAGAGTCTGGCCCTGGACTGGTGGCCCCAAGCCAGTCTCTGAGCATCACATGT
ACCGTGTCCGGCTTCAGCCTGACCAACTATGGCGTGCACTGGGTCCGACAGCCTCCAGGCAAA
GGACTGGAATGGCTGGGAGTGATTTGGGCTGGCGGCAGCACCAACTACAACAGCGCCCTGATG
AGCCGGCTGAGCATCTCCAAGGACAACAGCAAGAGCCAGGTGTTCCTGAAGATGAACAGCCTG
CAGACCGACGACACCGCCATGTACTACTGTGCTAGCAGAGGCGGCAACTACGGCTACGCCCTG
GATTATTGGGGCCAGGGCACAAGCGTGACCGTGTCATCT
SEQ ID NO: 21. Codon-optimized GD2 scFv-DNA sequence VH/VL format:
ATGGACTGGATCTGGCGGATTCTGTTTCTCGTGGGAGCTGCCACAGGCGCTCATTCTGCTCAGC
CTGCCCAGGTGCAAGTGAAAGAGTCTGGCCCTGGACTGGTGGCCCCAAGCCAGTCTCTGAGCA
TCACATGTACCGTGTCCGGCTTCAGCCTGACCAACTATGGCGTGCACTGGGTCCGACAGCCTCC
AGGCAAAGGACTGGAATGGCTGGGAGTGATTTGGGCTGGCGGCAGCACCAACTACAACAGCGC
CCTGATGAGCCGGCTGAGCATCTCCAAGGACAACAGCAAGAGCCAGGTGTTCCTGAAGATGAAC
AGCCTGCAGACCGACGACACCGCCATGTACTACTGTGCTAGCAGAGGCGGCAACTACGGCTAC
GCCCTGGATTATTGGGGCCAGGGCACAAGCGTGACCGTGTCATCTGGCGGCGGAGGAAGCGG
AGGCGGAGGATCTGGGGGCGGAGGCTCTGGCGGAGGGGGATCTAGCATCGTGATGACCCAGA
CTCCTAAGTTCCTGCTGGTGTCTGCCGGCGACAGAGTGACCATCACCTGTAAAGCCAGCCAGAG
CGTGTCCAACGACGTGGCCTGGTATCAGCAGAAGCCTGGACAGAGCCCCAAGCTGCTGATCTA
CAGCGCCAGCAACAGATACACCGGCGTGCCCGATAGATTCACCGGCTCTGGCTACGGCACCGA
CTTCACCTTTACCATCAGCACCGTGCAGGCCGAGGATCTGGCCGTGTACTTCTGCCAGCAAGAC
TACAGCTCTCTCGGCGGAGGCACCAAGCTGGAAATCAAA
ATG = start codon
SEQ ID NO: 22. GD2 scFv-Protein sequence VL/VH format:
MDWIWRILFLVGAATGAHSAQPASIVMTQTPKFLLVSAGDRVTITCKASQSVSNDVAWYQQKPGQSP
KLLIYSASNRYTGVPDRFTGSGYGTDFTFTISTVQAEDLAVYFCQQDYSSLGGGTKLEIKGGGGSGG
GGSGGGGSGGGGSQVQVKESGPGLVAPSQSLSITCTVSGFSLTNYGVHWVRQPPGKGLEWLGVI
WAGGSTNYNSALMSRLSISKDNSKSQVFLKMNSLQTDDTAMYYCASRGGNYGYALDYWGQGTSVT
VSS
SEQ ID NO: 23. GD2 scFv-Protein sequence VH/VL format:
MDWIWRILFLVGAATGAHSAQPAQVQVKESGPGLVAPSQSLSITCTVSGFSLTNYGVHWVRQPPGK
GLEWLGVIWAGGSTNYNSALMSRLSISKDNSKSQVFLKMNSLQTDDTAMYYCASRGGNYGYALDY
WGQGTSVTVSSGGGGSGGGGSGGGGSGGGGSSIVMTQTPKFLLVSAGDRVTITCKASQSVSNDVA
WYQQKPGQSPKWYSASNRYTGVPDRFTGSGYGTDFTFTISTVQAEDLAVYFCQQDYSSLGGGTKL
EIK
SEQ ID NO: 24. High Affinity Variant Immunoglobulin Gamma Fc Region Receptor III-A amino acid
sequence (full length form).
Met Trp Gln Leu Leu Leu Pro Thr Ala Leu Leu Leu Leu Val Ser Ala Gly Met Arg Thr Glu Asp Leu Pro
Lys Ala Val Val Phe Leu Glu Pro Gln Trp Tyr Arg Val Leu Glu Lys Asp Ser Val Thr Leu Lys Cys Gln
Gly Ala Tyr Ser Pro Glu Asp Asn Ser Thr Gln Trp Phe His Asn Glu Ser Leu Ile Ser Ser Gln Ala Ser
Ser Tyr Phe Ile Asp Ala Ala Thr Val Asp Asp Ser Gly Glu Tyr Arg Cys Gln Thr Asn Leu Ser Thr Leu
Ser Asp Pro Val Gln Leu Glu Val His Ile Gly Trp Leu Leu Leu Gln Ala Pro Arg Trp Val Phe Lys Glu
Glu Asp Pro Ile His Leu Arg Cys His Ser Trp Lys Asn Thr Ala Leu His Lys Val Thr Tyr Leu Gln Asn
Gly Lys Gly Arg Lys Tyr Phe His His Asn Ser Asp Phe Tyr Ile Pro Lys Ala Thr Leu Lys Asp Ser Gly
Ser Tyr Phe Cys Arg Gly Leu Val Gly Ser Lys Asn Val Ser Ser Glu Thr Val Asn Ile Thr Ile Thr Gln Gly
Leu Ala Val Ser Thr Ile Ser Ser Phe Phe Pro Pro Gly Tyr Gln Val Ser Phe Cys Leu Val Met Val Leu
Leu Phe Ala Val Asp Thr Gly Leu Tyr Phe Ser Val Lys Thr Asn Ile Arg Ser Ser Thr Arg Asp Trp Lys
Asp His Lys Phe Lys Trp Arg Lys Asp Pro Gln Asp Lys
SEQ ID NO: 25. High Affinity Variant Immunoglobulin Gamma Fc Region Receptor III-A nucleic acid
sequence (full length form).
ATGTGGCA GCTGCTGCTG CCTACAGCTC TCCTGCTGCT GGTGTCCGCC GGCATGAGAA
CCGAGGATCT GCCTAAGGCC GTGGTGTTCC TGGAACCCCA GTGGTACAGA GTGCTGGAAA
AGGACAGCGT GACCCTGAAG TGCCAGGGCG CCTACAGCCC CGAGGACAAT AGCACCCAGT
GGTTCCACAA CGAGAGCCTG ATCAGCAGCC AGGCCAGCAG CTACTTCATCGACGCCGCCA
CCGTGGACGA CAGCGGCGAG TATAGATGCC AGACCAACCT GAGCACCCTGAGCGACCCCG
TGCAGCTGGA AGTGCACATC GGATGGCTGC TGCTGCAGGC CCCCAGATGGGTGTTCAAAG
AAGAGGACCC CATCCACCTG AGATGCCACT CTTGGAAGAA CACCGCCCTGCACAAAGTGA
CCTACCTGCA GAACGGCAAG GGCAGAAAGT ACTTCCACCA CAACAGCGAC TTCTACATCC
CCAAGGCCAC CCTGAAGGAC TCCGGCTCCT ACTTCTGCAG AGGCCTCGTGGGCAGCAAGA
ACGTGTCCAG CGAGACAGTG AACATCACCA TCACCCAGGG CCTGGCCGTGTCTACCATCA
GCAGCTTTTT CCCACCCGGC TACCAGGTGT CCTTCTGCCT CGTGATGGTGCTGCTGTTCG
CCGTGGACAC CGGCCTGTAC TTCAGCGTGA AAACAAACAT CAGAAGCAGCACCCGGGACT
GGAAGGACCA CAAGTTCAAG TGGCGGAAGG ACCCCCAGGA CAAGTGA
SEQ ID NO: 26. CD8 Hinge Region amino acid sequence (Human)
LSNSIMYFSHFVPVFLPAKPTTTPAPRPPTPAPTIASQPLSLRPEACRPAAGGAVHTRGLD
SEQ ID NO: 27. CD8a Hinge Region DNA (Human)
CTGAGCAACAGCATCATGTACTTCAGCCACTTCGTGCCTGTGTTCCTGCCTGCCAAGCCTACAAC
AACACCAGCCCCTAGACCTCCAACCCCTGCCCCTACAATTGCCTCTCAGCCTCTGTCTCTGAGG
CCCGAAGCTTGTAGACCTGCTGCTGGCGGAGCTGTGCACACCAGAGGACTGGAT
SEQ ID NO: 28. Human T-cell surface glycoprotein CD3 zeta chain isoform 2 precursor
PKLCYLL DGILFIYGVI LTALFLRVKF SRSADAPAYQ QGQNQLYNEL NLGRREEYDV
LDKRRGRDPE MGGKPRRKNP QEGLYNELQK DKMAEAYSEI GMKGERRRGK GHDGLYQGLS
TATKDTYDAL HMQALPPR
End of Informal Sequence Listing

1. A method for inducing and maintaining an immune response to a tumor in a subject while treating a primary tumor, the method comprising administering to the subject an effective amount of CAR-expressing-NK-92 cells to treat the primary tumor thereby inducing an anti-tumor immune response that is maintained in the subject, the maintained immune response preventing tumor regrowth and/or inhibiting generation of secondary tumors.

2. The method of claim 1, wherein interleukin 6 expression is increased in the subject.

3. The method of claim 1, wherein the CAR-expressing-NK-92 cells induce lysis of tumor cells in the primary tumor.

4. The method of claim 1, wherein a cytokine is co-administered to the subject.

5. The method of claim 4, wherein the cytokine is interleukin 2.

6. The method of claim 4, wherein the cytokine is interleukin 12.

7. The method of claim 1, wherein a chemotherapeutic agent is administered to the subject prior to administration of the CAR-expressing-NK-92 cells.

8. The method of claim 1, wherein the CAR-expressing-NK-92 cells are administered systemically.

9. The method of claim 1, wherein the CAR-expressing-NK-92 cells are administered proximate to or directly into the primary tumor.

10. The method of claim 1, wherein the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor.

11. The method of claim 1, further comprising administering to the subject a cancer drug or radiation.

12. The method of claim 1, wherein the subject is selected from the group consisting of bovines, swine, rabbits, alpacas, horses, canines, felines, ferrets, rats, mice, fowl and buffalo.

13. The method of claim 1, wherein the subject is human.

14. The method of claim 1, wherein the CAR-expressing-NK-92 cells express a CD19-CAR on the cell surface.

15. The method of 14, wherein the CD19-CAR comprises an amino acid sequence at least 90% identical to SEQ ID NO: 3 or SEQ ID NO: 5.

16. A method of producing an anti-tumor vaccine in a subject with a tumor comprising administering to the subject an effective amount of CAR-expressing-NK-92 cells thereby inducing an anti-tumor vaccine to the tumor in the subject.

17. The method of claim 16, wherein interleukin-6 expression is increased in the subject.

18. The method of claim 16, wherein the CAR-expressing-NK-92 cells treats the tumor in the subject.

19. The method of claim 16, wherein a cytokine is co-administered to the subject.

20. The method of claim 19, wherein the cytokine is interleukin 2.

21. The method of claim 19, wherein the cytokine is interleukin 12.

22. The method of claim 16, wherein a chemotherapeutic agent is administered to the subject prior to administration of the CAR-expressing-NK-92 cells.

23. The method of claim 16, wherein the CAR-expressing-NK-92 cells are administered systemically.

24. The method of claim 16, wherein the CAR-expressing-NK-92 cells are administered proximate to or directly into the tumor.

25. The method of claim 16, wherein the tumor is selected from the group consisting of colorectal tumor, breast tumor, lung tumor, prostate tumor, pancreatic tumor, bladder tumor, cervical tumor, cholangiocarcinoma, gastric sarcoma, glioma, leukemia, lymphoma, melanoma, multiple myeloma, osteosarcoma, ovarian tumor, stomach tumor, brain tumor.

26. The method of claim 16, further comprising administering to the subject a cancer drug or radiation.

27. The method of claim 16, wherein the CAR-expressing-NK-92 cells express a CD19-CAR on the cell surface.

28. The method of 27, wherein the CD19-CAR comprises an amino acid sequence at least 90% identical to SEQ ID NO: 3 or SEQ ID NO: 5.

29. The method of claim 1, wherein the tumor is a B-cell lymphoma.

30.-32. (canceled)

33. The method of claim 1, wherein the CAR comprises an amino acid sequence at least 90% identical to SEQ ID NOs:3, 5, 7, 9, 11, 13, 15, 17, 19, 22, or 23.
